(19) United States
(12) Patent Application Publication
(10) Pub. No.: US 2023/0398058 A1
(43) Pub. Date: Dec. 14, 2023

(54) PARTICLES CONTAINING COLORING AGENTS AND METHODS OF USING THE SAME

(71) Applicant: Ephemeral Solutions, Inc., Milford, CT (US)

(72) Inventors: Brennal PIERRE, Bridgeport, CT (US); Vandan K. SHAH, Milford, CT (US); William CHIN, Rego Park, NY (US)

(21) Appl. No.: 18/230,410

(22) Filed: Aug. 4, 2023

Related U.S. Application Data
(63) Continuation of application No. PCT/US2022/016181, filed on Feb. 11, 2022.
(60) Provisional application No. 63/148,895, filed on Feb. 12, 2021.

(51) Int. Cl. A61K 8/90; A61K 8/02; A61Q 1/02

(52) U.S. Cl. CPC A61K 8/90; A61K 8/0233; A61Q 1/025; A61K 2800/596; A61K 2800/652; A61K 2800/434; A61K 2800/412; A61K 2800/91; A61K 2800/882

(57) ABSTRACT

This invention provides ink particles comprising a) a core comprising a coloring agent and a polymer, and b) an outermost layer comprising a surfactant or a dispersant, wherein the core further comprises an outer surface and the outermost layer at least partially covers the outer surface of the core. The invention further provides ink particles comprising a) a core comprising a coloring agent, b) a shell, and c) an outermost layer comprising a surfactant or a dispersant, wherein: the core or the shell comprises a polymer, wherein the polymer is polycaprolactone (PCL), poly D-lactic acid (PDLA), poly L-lactic acid (PLLA), poly(lactic-co-glycolic acid), (PLG), polyethylene glycol (PEG), polyethylene glycol-diacrylate (PEGDA), poly(sebacic anhydride) (poly(SA)), polyorthoester, aliphatic polyanhydride, aromatic polyanhydride, or a copolymer thereof; the core further comprises an outer surface; the shell at least partially covers the outer surface of the core; the shell further comprises an outer surface; and the outermost layer at least partially covers the outer surface of the shell. The invention also provides a composition of the ink particles and a liquid carrier or a kit comprising the ink particles and a liquid carrier, and methods of using the composition and the kit for use in a method for tattooing a subject.

### CROSS-REFERENCE TO RELATED APPLICATIONS

This application is a continuation of International Application No. PCT/2022/016181, filed Feb. 11, 2022, which claims the benefit of U.S. Provisional Application No. 63/148,895, filed Feb. 12, 2021, each of which is incorporated by reference herein in its entirety.

### FIELD OF THE INVENTION

This invention provides ink particles comprising a) a core comprising a coloring agent and a polymer, and b) an outermost layer comprising a surfactant or a dispersant, wherein the core further comprises an outer surface and the outermost layer at least partially covers the outer surface of the core. The invention further provides ink particles comprising a) a core comprising a coloring agent, b) a shell, and c) an outermost layer comprising a surfactant or a dispersant, wherein: the core or the shell comprises a polymer, wherein the polymer is polycaprolactone (PCL), poly D-lactic acid (PDLA), poly L-lactic acid (PLLA), poly(lactic-co-glycolic acid), (PLGA), polyethylene glycol (PEG), polyethylene glycol-diacrylate (PEGDA), poly(sebacic anhydride) (poly(SA)), polyorthoester, aliphatic polyanhydride, aromatic polyanhydride, or a copolymer thereof; the core further comprises an outer surface; the shell at least partially covers the outer surface of the core; the shell further comprises an outer surface; and the outermost layer at least partially covers the outer surface of the shell. The invention also provides a composition of the ink particles and a liquid carrier or a kit comprising the ink particles and a liquid carrier, and methods of using the composition and the kit for use in a method for tattooing a subject.

### BACKGROUND OF THE INVENTION

Evidence of skin ornamentation dates back to prehistoric times and have been used to signify status (e.g., marital status or military rank), to identify affiliations, and for aesthetic purposes. Tattooing has also been used therapeutically for treating dermatologic conditions, such as hypopigmentation and hyperpigmentation caused by vitiligo, skin grafts, and port-wine stains.

Permanent tattoos are typically applied by depositing ink into the dermis using a tattoo machine (e.g., a tattoo gun). Carriers for the pigment, e.g., water, are absorbed, and the insoluble pigment particles remain in the dermis where initially deposited. The inertness and aggregation results upon deposition of the tattoo ink particles prevent their elimination from the interstitial space of the tissue by the immune system, and therefore leads to its permanent effects.

Over the years, an individual's style, interests, and skin laxity may evolve. Although tattoos may be removed using laser-based methods, such methods are relatively expensive, painful and may not completely eliminate the tattoo. Additionally, surgical removal, dermabrasion, and salabrasion are invasive removal procedures and may lead to scarring. To avoid these drawbacks, some turn to paints that can be drawn on the skin (e.g. henna). These paints, however, are easily washed off and do not provide the receiver with the genuine feeling of having a somewhat permanent tattoo. The desire exists for semi-permanent tattoos that are applied the same way as the permanent tattoos, and can retain their vibrancy for about 1 month to about 18 months instead of being permanent.

### SUMMARY OF THE INVENTION

The present invention provides an ink particle comprising: (a) a core comprising a coloring agent and a polymer, wherein the polymer is polycaprolactone (PCL), poly D-lactic acid (PDLA), poly L-lactic acid (PLLA), poly(lactic-co-glycolic acid), (PLGA), polyethylene glycol (PEG), polyethylene glycol-diacrylate (PEGDA), poly(sebacic anhydride) (poly(SA)), polyorthoester, aliphatic polyanhydride, aromatic polyanhydride, or a copolymer thereof; and (b) an outermost layer comprising a surfactant or a dispersant, wherein the core further comprises an outer surface and the outermost layer at least partially covers the outer surface of the core.

The present invention further provides an ink particle comprising: (a) a core comprising a coloring agent; (b) a shell; and (c) an outermost layer comprising a surfactant or a dispersant, wherein: the core or the shell comprises a polymer, wherein the polymer is polycaprolactone (PCL), poly D-lactic acid (PDLA), poly L-lactic acid (PULA), poly(lac co-glycolic acid), (PLGA), polyethylene glycol (PEG), polyethylene glycol-diacrylate (PEGDA), poly(sebacic anhydride) (poly(SA)), polyorthoester, aliphatic polyanhydride, aromatic polyanhydride, or a copolymer thereof; the core further comprises an outer surface; the shell at least partially covers the outer surface of the core; the shell further comprises an outer surface; and the outermost layer at least partially covers the outer surface of the shell.

Each of the above ink particle is an “ink particle of the invention”.

The present invention further provides an ink composition comprising plurality of ink particles of the invention (each ink composition being an “ink composition of the invention”).

The present invention further provides a kit comprising a) the ink composition of the invention, and b) a liquid carrier.

The present invention further provides a kit comprising: a) an ink composition comprising plurality of ink particles, wherein each ink particle comprises a core comprising a coloring agent and a polymer, wherein the polymer is polycaprolactone (PCL), poly D-lactic acid (PDLA), poly L-lactic acid (PLLA), poly(lactic-co-glycolic acid), (PLGA), polyethylene glycol (PEG), polyethylene glycol-diacrylate (PEGDA), poly(sebacic anhydride) (poly(SA)), polyorthoester, aliphatic polyanhydride, aromatic polyanhydride, or a copolymer thereof; and b) a liquid carrier comprising a surfactant or a dispersant.

The present invention further provides a kit comprising: a) an ink composition comprising plurality of ink particles, wherein each ink particle comprises: (i) a core comprising a coloring agent; and (ii) a shell; wherein the core or the shell comprises a polymer, wherein the polymer is polycaprolactone (PCL), poly D-lactic acid (PDLA), poly L-lactic acid (PLLA), poly(lactic-co-glycolic acid), (PLGA), polyethylene glycol (PEG), polyethylene glycol-diacrylate (PEGDA), poly(sebacic anhydride) (poly(SA)), polyorthoester, aliphatic polyanhydride, aromatic polyanhydride, or a copolymer thereof; and b) a liquid carrier comprising a surfactant or a dispersant.

Each of the above kits is a “kit of the invention”.

The present invention further provides a method for tattooing a subject, comprising intradermally administering to the subject a cosmetically effective amount of the ink composition of the invention.

The present invention further provides a method for tattooing a subject, comprising the steps of: a) admixing the ink composition and the liquid carrier of the kit of the invention to provide a tattoo ink; and b) intradermally administering a cosmetically effective amount of the tattoo ink to the subject.

Each of the above methods is a “method of the invention”.

### BRIEF DESCRIPTION OF THE DRAWINGS

FIG. 1A (not to scale) shows the diameter (D) and the hydrodynamic diameter (Dh) of an illustrative ink particle of the invention comprising (a) a core comprising a coloring agent and a polymer and (b) an outermost layer comprising a surfactant or a dispersant.

FIG. 1B (not to scale) shows the diameter (D) and the hydrodynamic diameter (Dh) of an illustrative ink particle of the invention comprising (a) a core comprising a coloring agent, (b) a shell, and (c) an outermost layer comprising a surfactant or a dispersant.

FIG. 2A (not to scale) depicts an illustrative ink particle of the invention having a surfactant or dispersant on the outermost layer.

FIG. 2B (not to scale) depicts a cross section of an illustrative ink particle of the invention comprising a core and a outermost layer comprising a surfactant or a dispersant.

FIG. 2C (not to scale) depicts a cross section of an illustrative ink particle of the invention comprising a core, a shell, and a outermost layer comprising a surfactant or a dispersant.

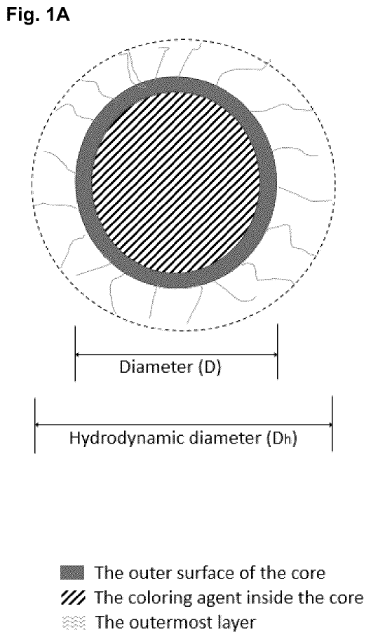

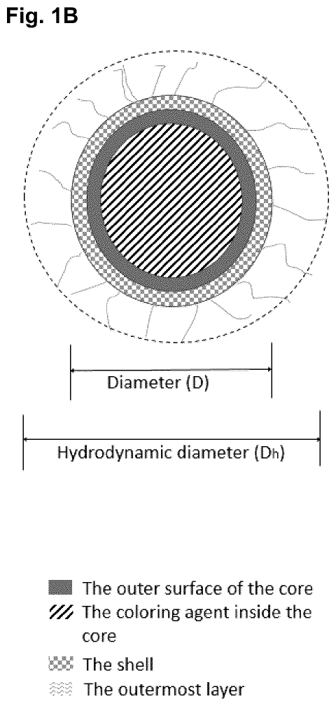

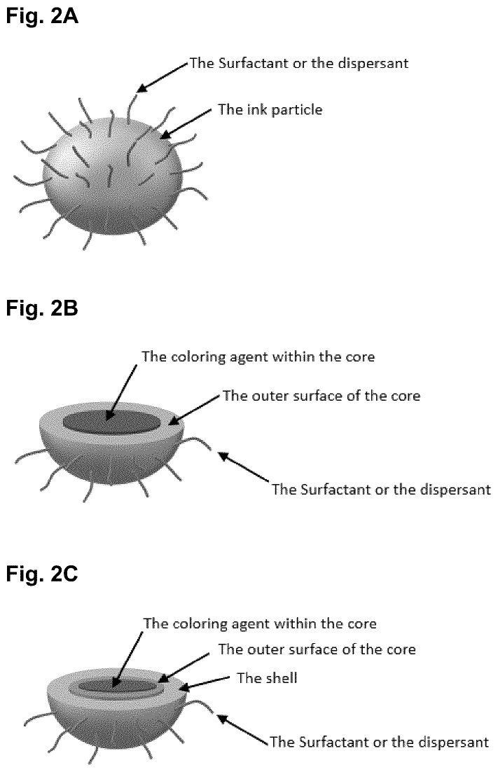

### DETAILED DESCRIPTION OF THE INVENTION

### Definitions

The term “about” when immediately preceding a numerical value means ± up to 20% of the numerical value. For example, “about” a numerical value means ± up to 20% of the numerical value, in some embodiments, ± up to 19%, ± up to 18%, ± up to 17%, ± up to 16%, ± up to 15%, ± up to 14%, ± up to 13%, ± up to 12%, ± up to 11%, ± up to 10%, ± up to 9%, ± up to 8%, ± up to 7%, ± up to 6%, ± up to 5%, ± up to 4%, ± up to 3%, ± up to 2%, ±up to 1%, ± up to less than 1%, or any other value or range of values therein.

Throughout the present specification, numerical ranges are provided for certain quantities. These ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

A “cosmetically effective amount” means an amount of the ink composition of the invention that, when intradermally administered to a subject is effective in a method of the invention.

A “subject” is a human or non-human mammal, e.g., a bovine, horse, feline, canine, rodent, or non-human primate. The human can be a male or female, child, adolescent or adult. The female can be premenarcheal or postmenarcheal.

“Mammal” includes a human, domestic animal such as a laboratory animal (e.g., mouse, rat, rabbit, monkey, dog, etc.) and household pet (e.g., cat, dog, swine, cattle, sheep, goat, horse, rabbit), livestock, and a non-domestic, wild animal.

All weight percentages (i.e., “% by weight” and “wt. %” and w/w) referenced herein, unless otherwise indicated, are relative to the total weight of the ink particle or the ink composition, as the case may be.

As used herein, a “biodegradation profile” or a “bioabsorption profile” of the ink composition of the invention refers to the biodegradation or the bioabsorption characteristics, respectively, with time of the ink composition once the ink composition is intradermally administered to the subject. A “lag phase” refers to the time period after the ink composition of the invention is intradermally administered to the subject, where the ink particles of the invention does not show substantial biodegradation or bioabsorption, e.g., the biodegradation or the bioabsorption of the ink particles are very slow or scarcely appreciable. In some embodiments, during the lag phase, a tattoo created with the ink composition of the invention substantially retains its color and image.

The term “pigment disorder” as used herein, refers to a disorder involving skin pigment (e.g., melanin). Examples of pigment disorders include, but are not limited to, all forms of albinism, melasma, pigment loss after skin damage, vitiligo, and any dysfunctional pigment secretion by the skin. In some embodiments, the pigment disorder is a disorder of excess pigmentation.

The Ink Particles of the Invention

The present invention provides an ink particle comprising: (a) a core comprising a coloring agent and a polymer, wherein the polymer is polycaprolactone (PCL), poly D-lactic acid (PDLA), poly L-lactic acid (PLLA), poly(lactic-co-glycolic acid), (PLGA), polyethylene glycol (PEG), polyethylene glycol-diacrylate (PEGDA), poly(sebacic anhydride) (poly(SA)), polyorthoester, aliphatic polyanhydride, aromatic polyanhydride, or a copolymer thereof; and (b) an outermost layer comprising a surfactant or a dispersant, wherein the core further comprises an outer surface and the outermost layer at least partially covers the outer surface of the core.

The present invention further provides an ink particle comprising: (a) a core comprising a coloring agent; (b) a shell; and (c) an outermost layer comprising a surfactant or a dispersant, wherein: the core or the shell comprises a polymer, wherein the polymer is polycaprolactone (PCL), poly D-lactic acid (PDLA), poly L-lactic acid (MLA), poly(lactic-co-glycolic acid), (PLGA), polyethylene glycol (PEG), polyethylene glycol-diacrylate (PEGDA), poly(sebacic anhydride) (poly(SA)), polyorthoester, aliphatic polyanhydride, aromatic polyanhydride, or a copolymer thereof; the core further comprises an outer surface; the shell at least partially covers the outer surface of the core; the shell further comprises an outer surface; and the outermost layer at least partially covers the outer surface of the shell.

In some embodiments of the ink particles of the invention, the shell comprises the polymer. In some embodiments of the ink particles of the invention, the core comprises the polymer. In some embodiments of the ink particles of the invention, the shell and the core comprises the polymer, wherein the polymer of the shell and of the core are the same. In some embodiments of the ink particles of the invention, the shell and the core comprises the polymer, wherein the polymer of the shell and of the core are different.

In some embodiments of the ink particles of the invention, the polymer is a block copolymer. In some embodiments, the block copolymer is a diblock copolymer or a triblock copolymer

In some embodiments of the ink particle of the invention, the polymer is present in the ink particle at a concentration of about 10% w/w to about 90% w/w (% weight of the polymer relative to the total weight of the ink particle). In some embodiments, the polymer is present in the ink particle at a concentration of about 10% w/w, about 15% w/w, about 20% w/w, about 25% w/w, about 30% w/w, about 35% w/w, about 40% w/w, about 45% w/w, about 50% w/w, about 55% w/w, about 60% w/w, about 65% w/w, about 70% w/w, about 75% w/w, about 80% w/w, about 85% w/w, or about 90% w/w. In some embodiments, the polymer is present in the ink particle at a concentration of about 20% w/w to about 90% w/w, about 30% w/w to about 90% w/w, about 40% w/w to about 90% w/w, about 50% w/w to about 90% w/w, about 10% w/w to about 80% w/w, about 10% w/w to about 70% w/w, about 10% w/w to about 60% w/w, about 10% w/w to about 50% w/w, about 20% w/w to about 80% w/w, about 30% w/w to about 70% w/w, or about 40% w/w to about 60% w/w.

In some embodiments of the ink particles of the invention, the coloring agent is adsorbed to, physically entrapped by, ionically bonded to or covalently bonded to the polymer. In some embodiments, the coloring agent is entrapped by, encased by, complexed by, incorporated into, or encapsulated by the polymer.

In some embodiments of the ink particles of the invention, the coloring agent is adsorbed to, physically entrapped by, ionically bonded to or covalently bonded to the core. In some embodiments, the coloring agent is entrapped by, encased by, complexed by, incorporated into, or encapsulated by the core.

In some embodiments of the ink particles of the invention, the coloring agent is adsorbed to, physically entrapped by, ionically bonded to or covalently bonded to the shell. In some embodiments, the coloring agent is entrapped by, encased by, complexed by, incorporated into, or encapsulated by the shell.

In some embodiments of the ink particles of the invention, the polymer is biodegradable and/or bioabsorbable.

In some embodiments of the ink particles of the invention, the comprises two or more of the following polymers: polycaprolactone (PCL), poly D-lactic acid (PDLA), poly L-lactic acid (PLLA), poly(lactic-co-glycolic acid), (PLGA), polyethylene glycol (PEG), polyethylene glycol-diacrylate (PEGDA), poly(sebacic anhydride) (poly(SA)), polyorthoester, aliphatic polyanhydride, aromatic polyanhydride, or a copolymer thereof.

In some embodiments of the ink particles of the invention, the polymer is an aliphatic polyanhydride or an aromatic polyanhydride, wherein the aliphatic polyanhydride is poly(sebacic anhydride) and wherein the aromatic polyanhydride is poly[bis(p-carboxyphenoxy)methane)], poly[1,3-bis(p-carboxyphenoxy)propane)], poly[1,6-bis(p-carboxyphenoxy)hexane], poly[1,4-bis(hydroxyethypterephthalate-alt-ethyloxyphosphate], or poly[1,4-bis(hydroxyethypterephthalate-alt-ethyloxyphosphate]-co-1,4-bis(hydroxyethypterephthalate-co-terephthalate (80/20). In some embodiments, the polymer of the shell or the core is an aliphatic polyanhydride or an aromatic polyanhydride, wherein the aliphatic polyanhydride is poly(sebacic anhydride) and wherein the aromatic polyanhydride is poly[bis(p-carboxyphenoxy)methane)], poly[1,3-bis(p-carboxyphenoxy)propane)], poly[1,6-bis(p-carboxyphenoxy)hexane], poly[1,4-bis(hydroxyethypterephthalate-alt-ethyloxyphosphate], or poly[1,4-bis(hydroxyethyl)terephthalate-alt-ethyloxyphosphate]-co-1,4-bis(hydroxyethyl)terephthalate-co-terephthalate (80/20). In some embodiments, the polymer is a polyorthoester (POE), wherein the POE is POE I, POE II, POE III, or POE IV. In some embodiments, the polymer of the shell or the core is a polyorthoester (POE), wherein the POE is POE I, POE II, POE III, or POE IV.

In some embodiments of the ink particles of the invention, the polymer is biodegradable or bioabsorbable. Among the bioabsorbable, bioerodable, or biodegradable polymers which can be used are those disclosed in Higuchi et al, U.S. Pat. Nos. 3,981,303, 3,986,510, and 3,995,635, including zinc alginate poly(lactic acid), poly(vinyl alcohol), polyanhydrides, and poly(glycolic acid). Alternatively, microporous polymers are suitable, including those disclosed in Wong, U.S. Pat. No. 4,853,224, such as polyesters and polyethers, and Kaufman, U.S. Pat. Nos. 4,765,846 and 4,882,150. Other polymers which degrade slowly in vivo are disclosed in Davis et ah, U.S. Pat. No. 5,384,33.

In some embodiments of the ink particles of the invention, the coloring agent is entrapped by or is within the polymer. In some embodiments of the ink particles of the invention, the coloring agent is incorporated in the polymer. In some embodiments, the coloring agent is incorporated in the polymer by a method comprising including the coloring agent in a pre-polymer mixture or mixture of monomers, followed by polymerization. In some embodiments, the polymerization is an emulsion polymerization process. In some embodiments, the coloring agent is incorporated in the polymer by a method comprising dissolving or suspending the polymer and the coloring agent in a solvent, followed by evaporating the solvent. In some embodiments, evaporation of the solvent is a single or double emulsion solvent evaporation process. In some embodiments, the coloring agent is incorporated in the polymer by a method comprising melting the polymer and dissolving or suspending the coloring agent in the resultant melted polymer. In some embodiments, the above methods can be used to incorporate coloring agents in polymers to form layerless particles and/or particle shells.

In some embodiments of the ink particles of the invention, the coloring agent has a molecular weight of about 5 Daltons to about 10×10⁶ Daltons. In some embodiments, the coloring agent's rate of release can depend on the rate of the polymer bioabsorption. In some embodiments, the coloring agent's rate of the release can depend on a rate of diffusion. In some embodiments, the coloring agent's rate of the release can depend on ionic exchange.

In some embodiments of the ink particles of the invention, the coloring agent is a dye, is a pigment, is fluorescent, or is phosphorescent.

In some embodiments of the ink particles of the invention, the coloring agent is melanin, [Phthalocyaninato(2-)] copper, FD&C Red 40 (Food Red 17), FD&C Yellow 5, Nigrosin, Reactive Black 5, Acid Blue 113, Brilliant black BN Granular (Food Black 1), D&C Yellow 10, FD&C Blue 1 (Food Blue 2), FD&C Blue 2, Acid Black 1, Acid Black 24, Acid Black 172, Acid Black 194, Acid Black 210, Spirulina Extract Powder, Gardenia Yellow 98%, Gardenia Yellow 40%, Gardenia Black, Gardenia Blue, Gardenia Red, Cochineal/Carmine, Annatto, Beta carotene, D&C Orange 4, D&C Red 33, D&C Red 22, Ext D&C Violet 2, D&C Yellow 8, FD&C Green 3, FD&C Red 4, FD&C Yellow 6, FD&C Red 3, Ponceau 4R, Acid Red 52, Carmoisine, Amamath, Brown HT, Black PN, Green S, Patent Blue V, Tartrazine, Sunset Yellow, Quinolline Yellow, Erythrosine, Brilliant Blue, Indigo Carmine, D&C Green 5, D&C Red 17, D&C Red 21, D&C Red 27, D&C Yellow 11, D&C Violet 2, D&C Green 6, D&C Red 30, D&C Red 31, D&C Red 28, D&C Red 7, D&C Red 6, D&C Red 34, D&C Yellow 10, Fake of Carmoisine, Fake of Ponceau 4R, Fanchon Yellow, Toluidine Red, Fake of Acid red 52, Fake of Allura Red, Fake of Tartrazine, Fake of Sunset Yellow, Fake of Brilliant Blue, Fake of Erythrosine, Fake of Quinoline, Fake of Indigo Carmine, Fake Patent Blue V, Fake Black PN, Fithol Rubin B, Iron Oxide Red, Iron Oxide Yellow, Iron Oxide Black, Iron Blue, Titanium Dioxide, D&C Red 36, Carbon Black, Ultramarine Blue, Ultramarine Violet, Ultramarine Red/Pink, Chromium Oxide Green, Mica, Chromium Hydroxide Green, Talc, Manganese Violet, Iron Oxide Burgundy, Iron Oxide Sienna, Iron Oxide Tan, Iron Oxide Amber, Iron Oxide Brown-G, Iron Oxide Brown S, Sodium Copper Chlorophyllin, Caramel, Riboflavin, Canthaxanthin, Paprika, natural tumeric, D&C Green 8, Ext D&C Yellow 7, NOIR Brilliant BN, Ferric Ammonium Ferrocyanide, D&C Yellow 10 Fake, FD&C Yellow 5 Fake, FD&C Yellow 6 Fake, D&C Red 21 Fake, D&C Red 33 Fake, FD&C Red 40 Fake, D&C Red 27 Fake, D&C Red 28 Fake, FD&C Blue 1 Fake, D&C Red 30 Fake, D&C Red 36 Fake, D&C Red 6 Fake, D&C Red 7 Fake, or D&C Black 2, or a combination thereof.

In some embodiments of the ink particle of the invention, the coloring agent is present in the ink particle at a concentration of about 10% w/w to about 70% w/w (% weight of the coloring agent relative to the total weight of the ink particle). In some embodiments, the coloring agent is present in the ink particle at a concentration of about 25% w/w to about 50% w/w. In some embodiments, the coloring agent is present in the ink particle at a concentration of about 25% w/w to about 35% w/w. In some embodiments, the coloring agent is present in the ink particle at a concentration of about 10% w/w, about 15% w/w, about 20% w/w, about 25% w/w, about 30% w/w, about 35% w/w, about 40% w/w, about 45% w/w, about 50% w/w, about 55% w/w, about 60% w/w, about 65% w/w, or about 70% w/w. In some embodiments, the coloring agent is present in the ink particle at a concentration of about 20% w/w to about 60% w/w, about 25% w/w to about 55% w/w, about 25% w/w to about 50% w/w, about 30% w/w to about 50% w/w, about 35% w/w to about 50% w/w, or about 30% w/w to about 45% w/w.

In some embodiments of the ink particle of the invention, the coloring agent is present in a hydrogel matrix or vehicle. In some embodiments, coloring agent is incorporated in the hydrogen matrix or vehicle. In some embodiments, the hydrogel matrix or vehicle is formed by a method comprising cross-linking a polysaccharide or a mucopolysaccharide with a protein and loading the coloring agent into the hydrogel matrices. Proteins include both full-length proteins and polypeptide fragments, which in either case may be native, recombinantly produced, or chemically synthesized. Polysaccharides include both polysaccharides and mucopolysaccharides. A hydrogel in which the coloring agent can be incorporated to is disclosed in Feijen, U.S. Pat. No. 5,041,292, incorporated herein by reference. In some embodiments, the hydrogel comprises a protein, a polysaccharide, and a cross-linking agent, providing network linkages therebetween wherein the weight ratio of polysaccharide to protein in the matrix ranges from about 10:90 to about 90:10. In some embodiments, the coloring agent is mixed into this matrix in an amount effective to provide a tattoo when the hydrogel matrix containing particles are intradermally administered to the dermis.

Examples of suitable polysaccharides include heparin, fractionated heparins, heparan, heparan sulfate, chondroitin sulfate, and dextran, including compounds described in U.S. Pat. No. 4,060,081 to Yannas et al., incorporated herein by reference. In some embodiments, the polysaccharide is heparin or a heparin analog. The protein component of the hydrogel may be either a full-length protein or a polypeptide fragment. The protein may be in native form, recombinantly produced, or chemically synthesized. The protein component of the hydrogel may also be a mixture of full-length proteins and/or fragments. In some embodiments, the protein is albumin, casein, fibrinogen, gamma-globulin, hemoglobin, ferritin or elastin. The protein component of the hydrogel may also be a synthetic polypeptide, such as poly (a-amino acid) polyaspartic acid or polyglutamic acid. In some embodiments, the protein component of the hydrogel is albumin.

In forming hydrogels containing coloring agents, the polysaccharide or mucopolysaccharide and the protein can be dissolved in an aqueous medium, followed by addition of an amide bond-forming cross-linking agent. In some embodiments, the cross-linking agent is a carbodiimide, for example, a water-soluble diimide, e.g., N-(3-dimethylaminopropyl)-N-ethylcarbodiimide. In some embodiments, the cross-linking agent is added to an aqueous solution of the polysaccharide and protein at an acidic pH and a temperature of about 0° C. to 50° C., in some embodiments, from about 4 to about 37° C., and allowed to react for up to about 48 hours. The resultant hydrogel can then be isolated, typically by centrifugation, and washed with a suitable solvent to remove uncoupled material.

In some embodiments, a mixture of the polysaccharide or mucopolysaccharide and protein is treated with a cross-linking agent having at least two aldehyde groups to form Schiff-base bonds between the components. In some embodiments, the bonds are then reduced with an appropriate reducing agent to give stable carbon-nitrogen bonds.

In some embodiments, once the hydrogel is formed, it is loaded with the coloring agent by a method comprising immersing the hydrogel in a solution or dispersion of the coloring agent and then evaporating the solvent. In some embodiments, the loaded hydrogels are dried in vacuo under ambient conditions.

Examples of polymers useful in the preparation of the hydrogel matrix or vehicle include one or more of alginate, chitosan or an acid salt thereof, alginate in combination with chitosan hydrochloride, methacrylate modified hyaluronic acid (HA-MA), thiolated hyaluronic acid (HA-SH), poly(N-isopropylacrylamide) (PNIPAM), polyethylene glycol (PEG), polycaprolactone (PCL), poly L-lactic acid (PLLA), poly(lactic-co-glycolic acid) (PLGA), diblock or triblock copolymers in any combination of PCL, PLLA, PLGA or PEG, polyethylene glycol-diacrylate (PEGDA), polyorthoester, and/or aliphatic or aromatic polyanhydrides or aliphatic-aromatic homopolyanhydrides, such as poly[bis(p-carboxyphenoxy)methane)] (poly(CPM)), poly[1,3-bis(p-carboxyphenoxy)propane)] (poly(CPP)), poly[1,6-bis(p-carboxyphenoxy)hexane] (poly(CPH)), poly(sebacic anhydride) (poly(SA)), poly[1,4-bis(hydroxyethypterephthalate-alt-ethyloxyphosphate], and/or poly[1,4-bis(hydroxyethypterephthalate-alt-ethyloxyphosphate]-co-1,4-bis(hydroxyethyl)terephthalate-co-terephthalate (P(BHET-EOP/BHET), 80/20).

In some embodiments, the polymer is poly(N-isopropylacrylamide) (PNIPAM). In some embodiments, the polymer comprises, consists essentially of or consists of repeat units of N-isopropylacrylamide or its functionalized derivatives shown in Table 1.

TABLE 1

| Chain-end group | |
|---|---|
| Functionalized Poly- | |
| (N-isopropylacrylamide) | |
| (PNIPAM) | Formula |
| Poly(N-isopropylacrylamide) | 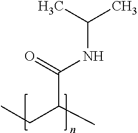 |
| Poly(N-isopropylacrylamide), carboxylic acid terminated | 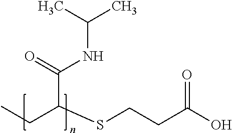 |
| Poly(N-isopropylacrylamide), amine terminated | 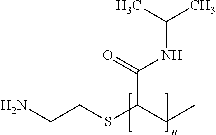 |
| Poly(N-isopropylacrylamide), azide terminated | 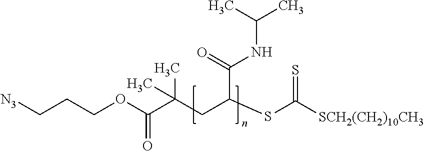 |
| Poly(N-isopropylacrylamide) triethoxysilane terminated | 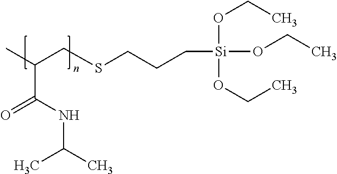 |
| Poly(N-isopropylacrylamide), maleimide terminated | 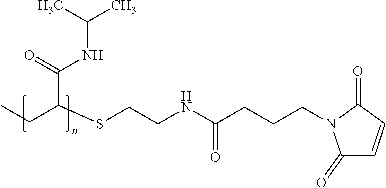 |
| Poly(N-isopropylacrylamide), N-hydroxysuccinimide (NHS) ester terminated | 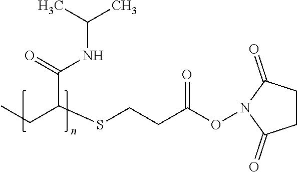 |

In some embodiments, the polymer is a copolymer of (i)N-isopropylacrylamide and, methacrylic acid or acrylic acid and (ii) a di-acrylamide crosslinker. In some embodiments, a copolymer of N-isopropylacrylamide has the structure of Formula I:

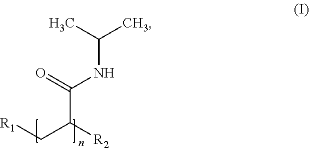

wherein:
R₁ is carboxy, hydroxyl, amino, or C₁ to C₃₀ alkyl, alkenyl, alkoxy, phenyl, cycloalkyl, phenoxy, aryl, or alkylamino,
and R₂ is carboxy, hydroxyl, amino, or C₁ to C30 alkyl, alkenyl, alkoxy, phenyl, cycloalkyl, phenoxy, aryl, or alkylamino.

In some embodiments of Formula (I), each R₁ and R₂ is independently a C₁ to C₂₅, C₁ to C₂₀, C₁ to C₁₅. C₁ to C₁₀, or C₁ to C₅ alkyl, alkenyl, alkoxy, phenyl, cycloalkyl, phenoxy, aryl, or alkylamino,

In some embodiments, the coloring agent is loaded into the hydrogel matrix or vehicle. In some embodiments, the hydrogel has a generally lower degree of cross-linking in the range of about 10 to about 2×10⁵ mol/m³.

In some embodiments, the hydrogels include activated heparin (i.e., heparin reacted with carbonyldiimidazole and saccharine) or heparin having one aldehyde group per molecule. In some embodiments, the hydrogels are treated with a positively charged macromolecular compound such as protamine sulfate, polylysine, or like polymers. In some embodiments, the hydrogel's surface is treated with a biodegradable block copolymer comprising, consisting essentially of or consisting of both hydrophilic and hydrophobic blocks. In some embodiments, the hydrophilic block is a positively charged polymer, such as polylysine. In some embodiments, the hydrophilic block is a biodegradable poly(a-amino acid), such as poly(L-alanine), poly(L-leucine), or a similar polymer.

In some embodiments, the coloring agent and an enzyme is encapsulated within a microcapsule core formed of a polymer which is specifically degraded by the enzyme. In some embodiments, the coloring agent and an enzyme is encapsulated within a microcapsule comprising of a core made up of a polymer around which there is an ionically-bound shell. In some embodiments, the polymer in the core of the microcapsule is an ionically cross-linked polysaccharide or calcium alginate, which is ionically coated with a polycationic skin of poly-L-lysine. In some embodiments, the enzyme is an alginase. In some embodiments, the Alginase is bacteria Beneckea pelagio or Pseudomonas putida. In some embodiments, the microcapsule core comprises chitin and the enzyme is chitinase. In some embodiments, the enzyme is hydrogenase.

In some embodiments of the ink particle of the invention, the ink particle has any shape or size. In some embodiments, the ink particle is spherical or non-spherical. For example, the ink particle is oblong or elongated, or have other shapes such as those disclosed in U.S. Patent Publication No. 2008/0112886; International Patent Publication No. WO 2008/031035; U.S. Patent Publication No. 2006/0201390; or U.S. Patent Publication No. 2007/0237800, each of which is incorporated herein by reference. If the ink particle is non-spherical, the ink particle may have a shape of, for instance, an ellipsoid, a cube, a fiber, a tube, a rod, or an irregular shape. In some embodiments, the ink particle is hollow or porous. Other shapes are also possible, including but not limited to, rectangular disk, high aspect ratio rectangular disk, high aspect ratio rod, worm, oblate ellipse, prolate ellipse, elliptical disk, UFO, circular disk, barrel, bullet, pill, pulley, biconvex lens, ribbon, ravioli, flat pill, bicone, diamond disk, emarginate disk, elongated hexagonal disk, taco, wrinkled prolate ellipsoid, wrinkled oblate ellipsoid, porous ellipsoid disk, substantially pyramidal, conical or substantially conical or the like.

In some embodiments of the ink particle of the invention, the ink particle has a diameter ranging from about 10 nm to about 100 μm. In some embodiments, the ink particle has a diameter ranging from about 10 nm to about 10 μm. In some embodiments, the ink particle has a diameter of about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 120 nm, about 140 nm, about 160 nm, about 180 nm, about 200 nm, about 220 nm, about 240 nm, about 60 nm, about 280 nm, about 300 nm, about 320 nm, about 340 nm, about 360 nm, about 380 nm, about 400 nm, about 420 nm, about 440 nm, about 460 nm, about 480 nm, about 500 nm, about 520 nm, about 540 nm, about 560 nm, about 580 nm, about 600 nm, about 620 nm, about 640 nm, about 660 nm, about 680 nm, about 700 nm, about 720 nm, about 740 nm, about 760 nm, about 780 nm, about 800 nm, about 820 nm, about 40 nm, about 860 nm, about 880 nm, about 900 nm, about 920 nm, about 40 nm, about 60 nm, about 980 nm, about 1 μm, about 5 μm, about 10 μm, about 20 μm, about 30 μm, about 40 μm, about 50 μm, about 60 μm, about 70 μm, about 80 μm, about 90 μm, or about 100 μm.

In some embodiments of the ink particles of the invention, a plurality of the ink particles have an average diameter ranging from about 10 nm to about 100 μm. In some embodiments, the ink particles have an average diameter ranging from about 10 nm to about 10 μm. In some embodiments, the ink particles have an average diameter of about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about nm, about 85 nm, about 90 nm, about 95 nm, about 100 nm, about 120 nm, about 140 nm, about 160 nm, about 180 nm, about 200 nm, about 220 nm, about 240 nm, about 60 nm, about 280 nm, about 300 nm, about 320 nm, about 340 nm, about 360 nm, about 380 nm, about 400 nm, about 420 nm, about 440 nm, about 460 nm, about 480 nm, about 500 nm, about 520 nm, about 540 nm, about 560 nm, about 580 nm, about 600 nm, about 620 nm, about 640 nm, about 660 nm, about 680 nm, about 700 nm, about 720 nm, about 740 nm, about 760 nm, about 780 nm, about 800 nm, about 820 nm, about 40 nm, about 860 nm, about 880 nm, about 900 nm, about 920 nm, about 40 nm, about 60 nm, about 980 nm, about 1 μm, about 5 μm, about 10 μm, about 20 μm, about 30 μm, about 40 μm, about 50 μm, about 60 μm, about 70 μm, about 80 μm, about 90 μm, or about 100 μm.

In some embodiments of the ink particles of the invention, the outermost layer covers from about 0.1% to about 99% of the outer surface of the core. In some embodiments, the outermost layer covers from about 1% to about 99%, from about 5% to about 99%, from about 10% to about 99%, from about 15% to about 99%, from about 20% to about 99%, from about 25% to about 99%, or from about 30% to about 99% of the outer surface of the core. In some embodiments, the outermost layer covers 100% of the outer surface of the core. In some embodiments, the outermost layer covers about 0.1%, about 0.2%, about 0.3%, about about 0.5%, about 0.6%, about 0.7%, about 0.8%, about 0.9%, about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about or about 99% of the outer surface of the core. In some embodiments, the outermost layer covers from about 30% to about 95%, from about 40% to about 95%, from about 50% to about 95%, from about 60% to about 95%, or from about 70% to about 95% of the outer surface of the core.

In some embodiments of the ink particles of the invention, the outermost layer covers from about 0.1% to about 99% of the outer surface of the shell. In some embodiments, the outermost layer covers from about 1% to about 99%, from about 5% to about 99%, from about 10% to about 99%, from about 15% to about 99%, from about 20% to about 99%, from about 25% to about 99%, or from about 30% to about 99% of the outer surface of the shell. In some embodiments, the outermost layer covers 100% of the outer surface of the shell. In some embodiments, the outermost layer covers about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about or about 99% of the outer surface of the shell. In some embodiments, the outermost layer covers from about 30% to about 95%, from about 40% to about 95%, from about 50% to about 95%, from about 60% to about 95%, or from about 70% to about 95% of the outer surface of the shell.

In some embodiments of the ink particle of the invention, the outermost layer increases the diameter of the ink particle by from about 1 nm to about 5000 nm with respect to the diameter of the core or, where the ink particle comprises a shell, the ink particle's core-plus-shell unit. In some embodiments, the outermost layer increases the diameter of the ink particle by about 1 nm, about 2 nm, about 3 nm, about 4 nm, about 5 nm, about 6 nm, about 7 nm, about 8 nm, about 9 nm, about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 120 nm, about 140 nm, about 160 nm, about 180 nm, about 200 nm, about 220 nm, about 240 nm, about 260 nm, about 280 nm, about 300 nm, about 320 nm, about 340 nm, about 360 nm, about 380 nm, about 400 nm, about 420 nm, about 440 nm, about 460 nm, about 480 nm, about 500 nm, about 520 nm, about 540 nm, about 560 nm, about 580 nm, about 600 nm, about 620 nm, about 640 nm, about 660 nm, about 680 nm, about 700 nm, about 720 nm, about 740 nm, about 760 nm, about 780 nm, about 800 nm, about 820 nm, about 840 nm, about 860 nm, about 880 nm, about 900 nm, about 920 nm, about 940 nm, about 960 nm, about 980 nm, about 1000 nm, about 1100 nm, about 1200 nm, about 1300 nm, about 1400 nm, about 1500 nm, about 1600 nm, about 1700 nm, about 1800 nm, about 1900 nm, about 2000 nm, about 2100 nm, about 2200 nm, about 2300 nm, about 2400 nm, about 2500 nm, about 2600 nm, about 2700 nm, about 2800 nm, about 2900 nm, about 3000 nm, about 3100 nm, about 3200 nm, about 3300 nm, about 3400 nm, about 3500 nm, about 3600 nm, about 3700 nm, about 3800 nm, about 3900 nm, about 4000 nm, about 4100 nm, about 4200 nm, about 4300 nm, about 4400 nm, about 4500 nm, about 4600 nm, about 4700 nm, about 4800 nm, about 4900 nm, or about 5000 nm with respect to the diameter of the ink particle before the application of the outermost layer. In some embodiments, the outermost layer is applied to increase the diameter of the ink particle, on average, from about 1 nm to about 3000 nm, from about 1 nm to about 1000 nm, from about 1 nm to about 800 nm, from about 1 nm to about 700 nm, from about 1 nm to about 600 nm, from about 1 nm to about 500 nm, from about 1 nm to about 400 nm, from about 1 nm to about 300 nm, from about 1 nm to about 200 nm, or from about 1 nm to about 100 nm with respect to the diameter of the ink particle before the application of the outermost layer. In some embodiments, the diameter is hydrodynamic diameter.

In some embodiments of the ink particle of the invention, the outermost layer has a thickness of from about 1 nm to about 5000 nm. In some embodiments, the outermost layer has a thickness of about 1 nm, about 2 nm, about 3 nm, about 4 nm, about 5 nm, about 6 nm, about 7 nm, about 8 nm, about 9 nm, about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 120 nm, about 140 nm, about 160 nm, about 180 nm, about 200 nm, about 220 nm, about 240 nm, about 260 nm, about 280 nm, about 300 nm, about 320 nm, about 340 nm, about 360 nm, about 380 nm, about 400 nm, about 420 nm, about 440 nm, about 460 nm, about 480 nm, about 500 nm, about 520 nm, about 540 nm, about 560 nm, about 580 nm, about 600 nm, about 620 nm, about 640 nm, about 660 nm, about 680 nm, about 700 nm, about 720 nm, about 740 nm, about 760 nm, about 780 nm, about 800 nm, about 820 nm, about 840 nm, about 860 nm, about 880 nm, about 900 nm, about 920 nm, about 940 nm, about 960 nm, about 980 nm, about 1000 nm, about 1100 nm, about 1200 nm, about 1300 nm, about 1400 nm, about 1500 nm, about 1600 nm, about 1700 nm, about 1800 nm, about 1900 nm, about 2000 nm, about 2100 nm, about 2200 nm, about 2300 nm, about 2400 nm, about 2500 nm, about 2600 nm, about 2700 nm, about 2800 nm, about 2900 nm, about 3000 nm, about 3100 nm, about 3200 nm, about 3300 nm, about 3400 nm, about 3500 nm, about 3600 nm, about 3700 nm, about 3800 nm, about 3900 nm, about 4000 nm, about 4100 nm, about 4200 nm, about 4300 nm, about 4400 nm, about 4500 nm, about 4600 nm, about 4700 nm, about 4800 nm, about 4900 nm, or about 5000 nm. In some embodiments, the outermost layer has a thickness from about 1 nm to about 3000 nm, from about 1 nm to about 1000 nm, from about 1 nm to about 800 nm, from about 1 nm to about 700 nm, from about 1 nm to about 600 nm, from about 1 nm to about 500 nm, from about 1 nm to about 400 nm, from about 1 nm to about 300 nm, from about 1 nm to about 200 nm, or from about 1 nm to about 100 nm. In some embodiments, the diameter is hydrodynamic diameter.

In some embodiments, an ink particle comprising a core and an outermost layer has a hydrodynamic diameter (Dh) that is greater than the ink particle's diameter (D) by from about 1 nm to about 1000 nm. In some embodiments, an ink particle comprising a core and an outermost layer has a hydrodynamic diameter (Dh) that is greater than the ink particle's diameter (D) by from about 1 nm to about 5000 nm. In some embodiments, the core has a hydrodynamic diameter and the outermost layer increases the hydrodynamic diameter by about 1 nm, about 2 nm, about 3 nm, about 4 nm, about 5 nm, about 6 nm, about 7 nm, about 8 nm, about 9 nm, about 10 nm, about 15 nm, about 20 nm, about 25 nm, about 30 nm, about nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 120 nm, about 140 nm, about 160 nm, about 180 nm, about 200 nm, about 220 nm, about 240 nm, about 260 nm, about 280 nm, about 300 nm, about 320 nm, about 340 nm, about 360 nm, about 380 nm, about 400 nm, about 420 nm, about 440 nm, about 460 nm, about 480 nm, about 500 nm, about 520 nm, about 540 nm, about 560 nm, about 580 nm, about 600 nm, about 620 nm, about 640 nm, about 660 nm, about 680 nm, about 700 nm, about 720 nm, about 740 nm, about 760 nm, about 780 nm, about 800 nm, about 820 nm, about 840 nm, about 860 nm, about 880 nm, about 900 nm, about 920 nm, about 940 nm, about 960 nm, about 980 nm, about 1000 nm, about 1100 nm, about 1200 nm, about 1300 nm, about 1400 nm, about 1500 nm, about 1600 nm, about 1700 nm, about 1800 nm, about 1900 nm, about 2000 nm, about 2100 nm, about 2200 nm, about 2300 nm, about 2400 nm, about 2500 nm, about 2600 nm, about 2700 nm, about 2800 nm, about 2900 nm, about 3000 nm, about 3100 nm, about 3200 nm, about 3300 nm, about 3400 nm, about 3500 nm, about 3600 nm, about 3700 nm, about 3800 nm, about 3900 nm, about 4000 nm, about 4100 nm, about 4200 nm, about 4300 nm, about 4400 nm, about 4500 nm, about 4600 nm, about 4700 nm, about 4800 nm, about 4900 nm, or about 5000 nm. In some embodiments, an ink particle comprising a core and an outermost layer has a hydrodynamic diameter (Dh) that is greater than the ink particle's diameter (D) by from about 1 nm to about 3000 nm, from about 1 nm to about 1000 nm, from about 1 nm to about 800 nm, from about 1 nm to about 700 nm, from about 1 nm to about 600 nm, from about 1 nm to about 500 nm, from about 1 nm to about 400 nm, from about 1 nm to about 300 nm, from about 1 nm to about 200 nm, or from about 1 nm to about 100 nm.

In some embodiments, an ink particle comprising a core, a and an outermost layer has a hydrodynamic diameter (Dh) that is greater than the ink particle's diameter (D) by from about 1 nm to about 1000 nm. In some embodiments, an ink particle comprising a core, a shell and an outermost layer has a hydrodynamic diameter (Dh) that is greater than the ink particle's diameter (D) by from about 1 nm to about 5000 nm. In some embodiments, the ink particle comprising a core, shell and the outermost layer has a hydrodynamic diameter (Dh) that is greater than the ink particle's diameter (D) by about 1 nm, about 2 nm, about 3 nm, about 4 nm, about 5 nm, about 6 nm, about 7 nm, about 8 nm, about 9 nm, about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 120 nm, about 140 nm, about 160 nm, about 180 nm, about 200 nm, about 220 nm, about 240 nm, about 260 nm, about 280 nm, about 300 nm, about 320 nm, about 340 nm, about 360 nm, about 380 nm, about 400 nm, about 420 nm, about 440 nm, about 460 nm, about 480 nm, about 500 nm, about 520 nm, about 540 nm, about 560 nm, about 580 nm, about 600 nm, about 620 nm, about 640 nm, about 660 nm, about 680 nm, about 700 nm, about 720 nm, about 740 nm, about 760 nm, about 780 nm, about 800 nm, about 820 nm, about 840 nm, about 860 nm, about 880 nm, about 900 nm, about 920 nm, about 940 nm, about 960 nm, about 980 nm, about 1000 nm, about 1100 nm, about 1200 nm, about 1300 nm, about 1400 nm, about 1500 nm, about 1600 nm, about 1700 nm, about 1800 nm, about 1900 nm, about 2000 nm, about 2100 nm, about 2200 nm, about 2300 nm, about 2400 nm, about 2500 nm, about 2600 nm, about 2700 nm, about 2800 nm, about 2900 nm, about 3000 nm, about 3100 nm, about 3200 nm, about 3300 nm, about 3400 nm, about 3500 nm, about 3600 nm, about 3700 nm, about 3800 nm, about 3900 nm, about 4000 nm, about 4100 nm, about 4200 nm, about 4300 nm, about 4400 nm, about 4500 nm, about 4600 nm, about 4700 nm, about 4800 nm, about 4900 nm, or about 5000 nm. In some embodiments, an ink particle comprising a core, shell and outermost layer has a hydrodynamic diameter (Dh) that is greater than the ink particle's diameter (D) by from about 1 nm to about 3000 nm, from about 1 nm to about 1000 nm, from about 1 nm to about 800 nm, from about 1 nm to about 700 nm, from about 1 nm to about 600 nm, from about 1 nm to about 500 nm, from about 1 nm to about 400 nm, from about 1 nm to about 300 nm, from about 1 nm to about 200 nm, or from about 1 nm to about 100 nm.

In some embodiments of the ink particle of the invention, the surfactant or the dispersant is present in the ink particle at a concentration of about 0.1% w/w to about 75% w/w (% weight of the surfactant or the dispersant relative to the total weight of the ink particle). In some embodiments, the surfactant or the dispersant is present in the ink particle at a concentration of about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1% w/w, about 2% w/w, about 3% w/w, about 4% w/w, about 5% w/w, about 6% w/w, about 7% w/w, about 8% w/w, about 9% w/w, about 10% w/w, about 15% w/w, about 20% w/w, about 25% w/w, about 30% w/w, about 35% w/w, about 40% w/w, about 45% w/w, about 50% w/w, about 55% w/w, about 60% w/w, about 65% w/w, about 70% w/w, or about 75% w/w. In some embodiments, the surfactant or the dispersant is present in the ink particle at a concentration of about 0.1% w/w to about 70% w/w, about 1% w/w to about 70% w/w, about 5% w/w to about 70% w/w, about 5% w/w to about 60% w/w, about 5% w/w to about 55% w/w, about 0.1% w/w to about 50% w/w, about 1% w/w to about 50% w/w, or about 5% w/w to about 50% w/w.

In some embodiments of the ink particles of the invention, the surfactant is a non-ionic surfactant, anionic surfactant, cationic surfactant, or amphoteric/zwitterionic surfactant, or the like, or a combination thereof.

In some embodiments of the ink particles of the invention, the surfactant is a non-ionic surfactant. In some embodiments, the non-ionic surfactant is poly(alkylene-oxide) block copolymer, oligomeric alkyl-ethylene oxide, alkyl-phenol poly-ethylene, sorbitan ester, or PEG-PLGA. In some embodiments, the non-ionic surfactant is PEG-PLGA, poloxamer, Tween, Span, or Pluronic. In some embodiments, the non-ionic surfactant is polyoxyethylene-polyoxypropylene or polyoxyethylene—polyoxypropylene—polyoxyethylene. In some embodiments, the non-ionic surfactant is a poloxamer. In some embodiments, poloxamer is poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, or poloxamer 407. In some embodiments, poloxamer is poloxamer 188 or poloxamer 407. In some embodiments, Tween is polysorbate 20 (molecular weight (MW)=1227.54 g/mol), polysorbate 40 (MW=1283.63 g/mol), polysorbate 60 (MW=1311.7 g/mol), or polysorbate 80 (MW=1310 g/mol). In some embodiments, Span is sorbitan monostearate, sorbitan tristearate, or sorbitan monolaurate. In some embodiments, Pluronic is Pluronic L61 (EO₂PO₃₀EO₂; MW=1,950 g/mol), Pluronic L62 (EO₆PO₃₄EO₆; MW=2,500 g/mol), Pluronic L64 (EO₁₃PO₃₀EO₁₃; MW=2,900 g/mol), Pluronic P65 (EO₁₉PO₂₉EO₁₉; MW=3,400 g/mol), Pluronic P84 (EO₀₉PO₄₃EO₁₉; MW=4,200 g/mol), Pluronic P85 (EO₂₅PO₄₀EO₂₅; MW=4,600 g/mol), Pluronic F88 (EO₁₀₃ PO₃₉EO₁₀₃; MW=11,400 g/mol), Pluronic P103 (EO₁₇PO₆₀EO₁₇; MW=4,950 g/mol), Pluronic P104 (EO₂₇PO₆₁EO₂₇; MW=5,900 g/mol, Pluronic P105 (EO₂₇PO₅₆EO₂₇; MW=6,500 g/mol), Pluronic F108 (EO₁₃₂PO₅₀EO₁₃₂; MW=14,600 g/mol), Pluronic P123 (EO₁₉PO₆₉EO₁₉; MW=5,800 g/mol), or Pluronic F127 (EO₉₇PO₆₉EO₉₇; MW=12,600 g/mol). EO=polyoxyethylene; PO=polyoxypropylene. In some embodiments, Pluronic is a poly(ethylene oxide)-poly(propylene oxide)-poly(ethylene oxide) triblock copolymer. In some embodiments, the non-ionic surfactant has the structure shown in Table 2. In some embodiments, the non-ionic surfactant is Triton X, Triton X (reduced), pentapropylene glycol monododecyl ether, octapropylene glycol monododecyl ether, Nonoxynol 9, glycerol monolaurate, pentaethylene glycol monododecyl ether, octaethylene glycol monododecyl ether, polyethoxylated tallow amine, poloxamer, lauramide monoethylamine, lauramide diethylamine, octyl glucoside, decyl glucoside, lauryl glucoside, digitonin, Tween, n-dodecyl-β-D-maltoside, Zonyl FSO, or 2,5-dimethyl-3-hexyne-2,5-diol.

TABLE 2
Non-Ionic Surfactants

| | | Common |
|---|---|---|
| | | Name, |
| | | Trade |
| | | Name, |
| Class of | | or |
| Compounds | Structure | Abbrev. |
| poly(alkylene- oxide) block copolymer | 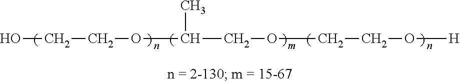 | Pluronic PEO- PPO- PEO |
| | 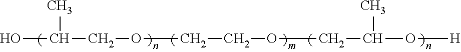 | Pluronic R PPO- PEO- |
| | | PPO |
| | 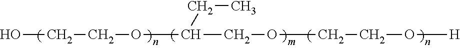 | PEO- PBO- PEO |
| | 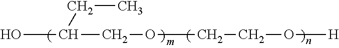 | PBO- PEO |
| | 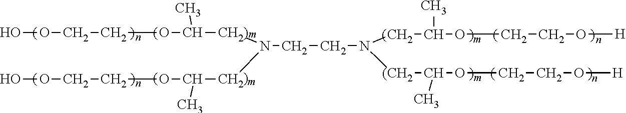 | Tetronic |
| oligomeric alkyl-ethylene | 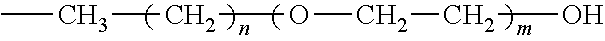 | Brij |
| oxide | | |
| | 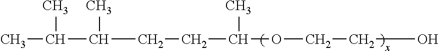 | Tergitol |
| alkyl-phenol poly-ethylene | 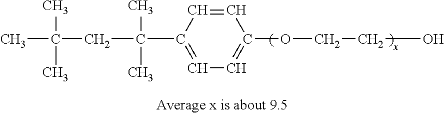 | Triton |
| sorbitan ester | 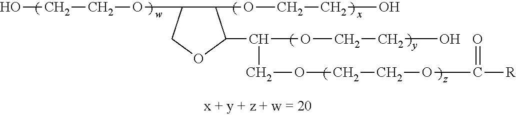 | Tween |
| | 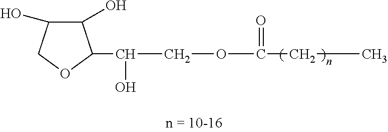 | Span |

PEO = poly(ethylene oxide); PPO = poly(propylene oxide); PBO = poly(n-butylene oxide)

In some embodiments of the ink particles of the invention, the surfactant is an anionic surfactant. In some embodiments, the anionic surfactant is sodium stearate, sodium lauroyl sarcosinate, cholic acid, deoxycholic acid, glycolic acid ethoxylate 4-tert-butylphenyl ether, glycolic acid ethoxylate laurylphenyl ether, glycolic acid ethoxylate oleyl ether, zonyl fluorosurfactant, sodium bis-2-ethylhexylsulphosuccinate (AOT), sodium dodecyl sulfate, sodium decyl sulfate, sodium N-lauroyl-N-methyltaurate, sodium tetradecyl sulfate, ammonium dodecyl sulfate, dioctyl sodium sulfosuccinate, sodium dodecylbenzenesulfonate, sodium lauryl ether sulfate, 3-sulfopropyl ethoxylate laurylphenyl ether, perfluorooctanesulfonic acid, perfluorobutanesulfonic acid, or sodium myreth sulfate. In some embodiments, glycolic acid ethoxylate 4-tert-butylphenyl ether has the following structure:

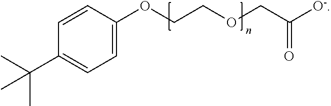

In some embodiments, glycolic acid ethoxylate laurylphenyl ether has the following structure:

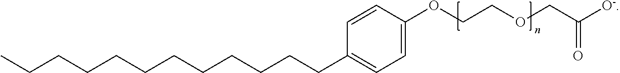

In some embodiments, glycolic acid ethoxylate oleyl ether has the following structure:

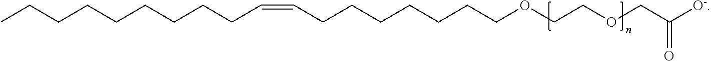

In some embodiments, zonyl fluorosurfactant has the following structure:

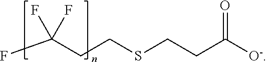

In some embodiments, sodium lauryl ether sulfate has the following structure:

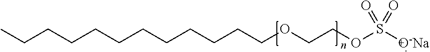

In some embodiments, 3-sulfopropyl ethoxylate laurylphenyl ether has the following structure:

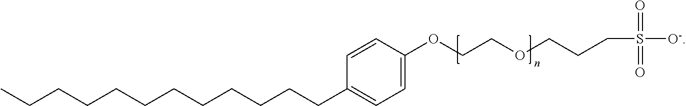

In some embodiments of the ink particles of the invention, the surfactant is a cationic surfactant. In some embodiments, the cationic surfactant is triethyalamine hydrochloride, octenidine dihydrochloride, cetrimonium bromide, cetylpyridinium chloride, benzalkonium chloride, benzethonium chloride, dimethyl dioctadecyl ammonium chloride, dioctadecyl dimethyl ammonium bromide, tetradecyltrimethylammonium bromide (TTAB), dodecyltrimethylanunonium bromide (DTAB), lecithin, diacylphosphatidylcholine, or quaternary ammonium alkyl salt. In some emboiments, quarternary ammonium alkyl salt is Adogen or dimethyldioctadecylammonium chloride. In some embodiments, Adogen is methyltrialkyl(C₈-C₁₀)ammonium chloride. In some embodiments, Adogen has the following structure:

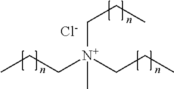

where n is 1-10.

In some embodiments of the ink particles of the invention, the surfactant is an amphoteric/zwitterionic surfactant. In some embodiments, the amphoteric/zwitterionic surfactant is 3[(3-Cholamidopropyl)dimethylammonio]-1-propanesulfonate (CHAPS), 3-[(3-cholamidopropyl)dimethylammonio]-2-hydroxy-1-propanesulfonate (CHAPSO), cocamidopropyl betaine, amidosulfobetaine-16, 3-(4-tert-butyl-1-pyridinio)-1-propanesulfonate, lauryl-N,N-(dimethyl)-glycinebetaine, 3-(1-pyridinio)-1-propanesulfonate, hexadecyl phosphocholine (Miltefosine), lauryl-N,N-(dimethyl)-propanesulfonate, 3-{N,N-Dimethyl-N-[3-(4-octylbenzoylamino)propyl]ammonio}propanesulfonate (C80 detergent), lauryldimethylamine N-oxide, tetradecyldimethylamine oxide (TDMAO) or phospholipid.

In some embodiments, the phospholipid is dipalmitoylphosphatidylcholine (DPPC), lecithin, distearoylphosphatidylcholine (DSPC) or dimyristoylphosphatidylcholine (DMPC).

In some embodiments of the ink particles of the invention, the dispersant is a naturally occurring polymer, synthetic polymer or semi-synthetic polymer. In some embodiments, the naturally occurring polymer is protein (e.g. albumin, cellulose, or an ester and or ether thereof), amylose, amylopectin, chitin, chitosan, collagen, gelatine, glycogen, polyamino acid (e.g. polylysine, starch, a dextran or heparin. In some embodiments, the synthetic polymer is polyethylene glycol (PEG), polyethyleneimine (PEI), polyvinyl alcohol (PVA), polyvinyl acetate, polyvinyl butyral, polyvinylpyrrolidone (PVP), polyacrylate, poloxamers or copolymer of PEG and polyester (PLGA, PCL, PLA) (e.g. PEG-PLGA), or the like. In some embodiments, the copolymer of PEG and polyester is a diblock or triblock copolymer. In some embodiments, the semi-synthetic polymer is a modified starch.

In some embodiments of the ink particles of the invention, the dispersant is nonionic (e.g., ethoxylated) or anionic (e.g., non-ethoxylated salt) such as that commercially available from Air Products and Chemicals, Inc. (e.g., SURFYNOL™ PSA336); Archer Daniels Midland Co. (e.g., ULTRALEC™ F deoiled lecithin); a sodium bis(2,6-dimethyl 4-heptyl)sulfosuccinate, such as that commercially available from Ashland Inc. (e.g., NEKAL™ WS-25-I); a fatty acid-modified emulsifier having a viscosity of 40,000 cps from BASF (e.g., DISPEX™ AA 4144 and DISPEX ULTRA FA 4425), a fatty acid-modified emulsifier and a dark brown liquid of unspecified viscosity from BASF (e.g., DISPEX ULTRA FA 4420), an aliphatic polyether with acidic groups having a viscosity of 350 cps from BASF (e.g., DISPEX ULTRA FA 4431), a fatty acid modified polymer having a viscosity of 10,000 cps from BASF (e.g., DISPEX ULTRA PA 4501), a modified polyurethane from BASF (e.g., DISPEX ULTRA PA 4510, EFKA™ PU 4010, EFKA PU 4047), a modified polyacrylate from BASF (e.g., EFKA PX 4300, EFKA ULTRA PA 4510 and EFKA ULTRA PA 4530), an acidic polyether having a viscosity of 1,400 cps from BASF (e.g., EFKA FA 4620), an unsaturated polyamide and acid ester salt having a viscosity of 2,000 cps from BASF (e.g., EFKA FA 4642), a difunctional block copolymer surfactants terminating in primary hydroxyl groups and having respective viscosities of 375, 450 and 600 cps from BASF (e.g., HYDROPALAT™ WE 3135, HYDROPALAT WE 3136 and HYDROPALAT WE 3317), a tetrafunctional block copolymer terminating in primary hydroxyl groups and having respective viscosities of 700 and 320 cps from BASF (e.g., TETRONIC™ 901 and TERTRONIC 904); a polyurethane oligomer having a viscosity of about 30,000 cps from Borchers (e.g., BORCHI™ Gen 0451), an amine neutralized acrylic acid copolymer having a viscosity of about 75-300 cps from Borchers (e.g., BORCHI Gen 0652), an acrylic ester copolymer having respective viscosities of about 1,500-3,500 and 50-300 cps from Borchers (e.g., BORCHI Gen 1252 and BORCHI Gen 1253); a solution of an ammonium salt of an acrylate copolymer from Byk-Chemie (e.g., BYK™ 156), a solution of an alkyl ammonium salt of a low-molecular-weight polycarboxylic acid polymer from Byk-Chemie (e.g., DISPERBYK™), an acidic copolymer from Byk-Chemie (e.g., DISPERBYK-102), a phosphoric ester salt of a high molecular copolymer with pigment affinic groups and a liquid of unspecified viscosity from Byk-Chemie (e.g., DISPERBYK™-145), a solution of a high molecular weight block copolymer with pigment affinic groups from Byk-Chemie (e.g., DISPERBYK-190), a structured copolymer with pigment affinic groups having a viscosity of 8,600 cps from Byk-Chemie (e.g., DISPERBYK-201), a copolymer with pigment affinic groups and a liquid of unspecified viscosity from Byk-Chemie (e.g., DISPERBYK-2055), a solution of a copolymer with pigment affinic groups having a viscosity of 3,600 cps from Byk-Chemie (e.g., DISPERBYK-2060), a solution of a copolymer with pigment affinic groups having a viscosity of 491 cps from Byk-Chemie (e.g., DISPERBYK-2061), a high molecular weight copolymer with pigment affinic groups sold in solid form as pastilles from Byk-Chemie (e.g., DISPERBYK-2091, DISPERBYK-2200), a copolymer with pigment affinic groups having a viscosity of 21,600 cps from Byk-Chemie (e.g., BYKJET™-9152), an aqueous solution of a novolac derivative having a viscosity of 4,000 cps from Clariant (e.g., DISPERSOGEN™ 1728), a novolac alkoxylate having a viscosity of 4,000 cps from Clariant (e.g., DISPEROGEN 2774), fatty alcohol ethoxylate having respective viscosities of about 400 cps and 1,300 cps from Clariant (e.g., GENAPOL™ X 1003 and GENAPOL X 1005), a sulfate ester having a viscosity of about 2,700 cps from Clariant (e.g., HOSTAPAL BV concentrate), an ammonia salt of a styrene maleic anhydride copolymer solution from Cray Valley (e.g., SMA1440H), a dispersant from Dow Chemical Co. (e.g., the TAMOL™ family including TAMOL 165A and TAMOL 731A), a dispersant from Elementis (e.g., NUOSPERSE™ FA196 which has a viscosity of 1,200 cps), a dispersant from Lubrizol (e.g., SOLSPERSE™ 27000, SOLSPERSE 28000, SOLSPERSE 32000, SOLSPERSE 39000, SOLSPERSE 64000, SOLSPERSE 65000, SOLSPERSE 66000, SOLSPERSE 71000, SOLSPERSE M387, SOLPLUS™ R700 and SOLPLUS K500), a dispersant from Ethox Chemicals, LLC (e.g., the E-SPERSE™ family and ETHOX™ 4658), a dispersant from Evonik (e.g., TEGO™ DISPERS 656, TEGO DISPERS 685, TEGO DISPERS 750W and TEGO DISPERS 757W), a nonionic surfactant from Rhodia Solvay Group (e.g., ABEX 2514 and ABEX 2525), an isopropyl amine dodecylbenzene sulfonate having a viscosity of 10,000 cps from Rhodia Solvay Group (e.g., RHODACAL™ IPAM), a polyoxyethylene tridecyl phosphate ester from Rhodia Solvay Group (e.g., RHODAFAC™ RS-710), a dispersant from Rhodia Solvay Group (e.g., RHODOLINE™ family including RHODOLINE 4170 and RHODOLINE 4188), a dispersant from Sasol Wax GmbH (e.g., ADSPERSE™ 100, ADSPERSE 500 and ADSPERSE 868), an isopropyl amine salt of an alkyl aryl sulfonate having a viscosity of about 6000 cps from Stepan Company (e.g., G-3300), a sodium dodecylbenzene sulfonate having a viscosity of about 85 cps from Stepan Company (e.g., POLYSTEP™ A-15), ethoxylated ammonium lauryl ether sulfates respectively containing 4 or 12 moles of ethylene oxide having respective viscosities of 66 and 42 cps from Stepan Company (e.g., POLYSTEP B-11 and POLYSTEP B-23), and sodium lauryl sulfate having a viscosity of 100 cps from Stepan Company (e.g., POLYSTEP B-24).

In some embodiments of the ink particles of the invention, the ink particle further comprises another polymer. In some embodiments, the other polymer is polyethylene, polystyrene, silicone, polyfluoroethylene, polyacrylic acid, a polyamide (e.g., nylon), polycarbonate, polysulfone, polyurethane, polybutadiene, polybutylene, polyethersulfone, polyetherimide, polyphenylene oxide, polymethylpentene, polyvinylchloride, polyvinylidene chloride, polyphthalamide, polyphenylene sulfide, polyester, polyetheretherketone, polyimide, polymethylmethacylate and/or polypropylene. In some embodiments, the ink particle further comprises a ceramic such as tricalcium phosphate, hydroxyapatite, fluorapatite, aluminum oxide, or zirconium oxide.

In some embodiments, the other polymer is a biocompatible and/or biodegradable polymer such as polylactic and/or polyglycolic acids, polyanhydride, polycaprolactone, polyorthoester, polyethylene oxide, polybutylene terephthalate, starch, cellulose, chitosan, and/or combinations of these. In some embodiments, the ink particle further comprises a hydrogel, such as agarose, collagen, or fibrin.

In some embodiments, the ink particle further comprises a magnetically susceptible material, e.g., a material displaying paramagnetism or ferromagnetism. For instance, the ink particle further comprises iron, iron oxide, magnetite, hematite, or some other compound comprising iron. In some embodiments, the ink particle further comprises a conductive material (e.g., a metal such as titanium, copper, platinum, silver, gold, tantalum, palladium, rhodium, etc.), or a semiconductive material (e.g., silicon, germanium, CdSe, CdS, etc.). In some embodiments, the ink particles further comprise ZnS, ZnO, TiO₂, AgI, AgBr, HgI₂, PbS, PbSe, ZnTe, CdTe, In₂S₃, In₂Se₃, Cd₃P₂, Cd₃As₂, InAs, or GaAs.

In some embodiments, the ink particle does not comprise a metal.

In some embodiments, the ink particle further comprises another species, such as a cell, biochemical specie such as nucleic acid (e.g., RNA, DNA, PNA, etc.), protein, peptide, enzyme, nanoparticle, quantum dot, fragrance, indicator, dye, fluorescent specie, chemical, small molecule (e.g., having a molecular weight of less than about 1 kDa).

In some embodiments of the ink particles of the invention, the ink particle is stable at ambient temperature for at least about 12 months. In some embodiments, the ink particle is stable at ambient temperature for at least about 6 months, at least about 12 months, at least about 18 months, or at least about 24 months. In some embodiments of the ink particles of the invention, the ink particle is stable at ambient temperature for at least about 12 months in the absence of moisture.

In some embodiments of the ink particles of the invention, the ink particle is stable when stored at −20° C. in less than 5% relative humidity (<5% RH) for at least about 12 months. In some embodiments of the ink particles of the invention, the ink particle is stable when stored at −20° C. in less than 5% relative humidity for at least about 1 year, at least about 2 years, at least about 3 years, at least about 4 years, or at least about 5 years.

In some embodiments of the ink particles of the invention, the ink particle is stable when stored using a dessicant and/or an airtight packaging for at least about 12 months. In some embodiments, the ink particle is stable when stored using a dessicant and/or an airtight packaging for at least about 6 months, at least about 12 months, at least about 18 months, at least about 24 months, at least about 30 months, or at least about 36 months. In some embodiments, the ink particles are stored at less than 5% relative humidity.

In some embodiments of the ink particles of the invention, the ink particle further comprises an additive. In some embodiments, the additive is succinylated gelatin, arabinogalactan, glutaraldehyde, or petroleum wax, or mixtures thereof. In some embodiments, the additive is poloxanele, poly(acrylic acid co-hypophosphorite) sodium salt, polyacrylamide, alginate/alginic acid, calcium caseinate, calcium polypectate, cellulose acetate phthalate, cellulose acetate trimellitate, chitosan, edible and natural waxes, fatty acids, fatty alcohols, gellan gums, hydroxy cellulose, hydroxy ethyl cellulose, hydroxy methyl cellulose, hydroxy propyl cellulose, hydro propyl ethyl cellulose, hydroxy propyl methyl cellulose phthalate, lipids, mono-, di- and triglycerides, pectins, phospholipids, polyalkyl(Ci6-C22) acrylate, polyethylene, oxidized polyethylene, polyethyleneimine reacted with 1,2-dichloroethane, polyoxyethylene(600)dioleate, polyoxyethylene(600)monoricinoleate, polyoxyethylene(23)lauryl ether, polyethylene glycol, polyethylene glycol(400)dioleate, polyethylene glycol(400)mono-& di-oleate, polyglycerol esters of fatty acids, polyisobutylene, polyglycerol phthalate ester of coconut oil fatty acids, polymaleic acid and/or its sodium salts, polyoxyethylene glycol(400)mono-& di-oleates, polyoxyethylene (23) lauryl ether, polyoxyethylene(40)monostearate, polyoxyethylene-polyoxypropylene block polymers, polyoxyethylene(20)sorbitan monooleate, polyoxyethylene (20) sorbitan monostearate, polyoxyethylene(2)sorbitan tristearate, polyoxypropylene glycol, polyvinyl acetate, polysorbate 80, polyvinylpolypyrrolidone, polyvinylpyrrolidone, or poly(20vinylpyridine-co-styrene). In some embodiments, the additive is wax, polyolefin, paraffin (e.g., Bayberry, spermaceti, Japan, Ross, etc.), triglyceride, phospholipid, fatty acid or ester thereof (e.g., lauric acid, palmitic acid, sorbitan monopalmitate, sorbitan monostearate, etc.), poly(vinyl palmitate), poly(hexadecyl acrylamide), poly(butyl acrylate), poly(hexadecyl acrylate), poly(octadecyl acrylate), poly(dodecene), poly(isobutene), poly(trimethyl glutarate), polyanhydrides, poly orthoesters, polyesters, polystyrene, polyurethane, polypropylene, polymethacrylate, polytetrafluoroethylene, ceramic, or glass. In some embodiments, the additive is present in the core. In some embodiments, the additive is present in the shell. In some embodiments, the additive is present in the outermost layer. In some embodiments, the additive is present in the core, the shell, and/or the outermost layer.

In some embodiments of the ink particles of the invention, the ink particle further comprises an aggregation agent. In some embodiments, the aggregation agent is an alkyl cyanoacrylate monomer. In some embodiments, the alkyl cyanoacrylate monomer is methyl cyanoacrylate, n-butyl cyanoacrylate, isobutyl cyanoacrylate, n-hexyl cyanoacrylate, 2-hexyl cyanoacrylate, 2-octyl cyanoacrylate, methoxyisopropyl cyanoacrylate, or a combination thereof. In some embodiments, the aggregation agent is present in the ink particle at a concentration of about 0.2% w/w to about 75% w/w, about 0.3% w/w to about 75% w/w, about 0.4% w/w to about 75% w/w, about 0.5% w/w to about 75% w/w, about 0.6% w/w to about 75% w/w, about 1% w/w to about 75% w/w, about 2% w/w to about 75% w/w, about 3% w/w to about 75% w/w, about 4% w/w to about 75% w/w, about 5% w/w to about 75% w/w, about 10% w/w to about 75% w/w, about 15% w/w to about 75% w/w, about 20% w/w to about 75% w/w, about 25% w/w to about 75% w/w, about 30% w/w to about 75% w/w, about 35% w/w to about 75% w/w, about 40% w/w to about 75% w/w, about 45% w/w to about 75% w/w, about 50% w/w to about 75% w/w, about 55% w/w to about 75% w/w, about 60% w/w to about 75% w/w, about 65% w/w to about 75% w/w, about 70% w/w to about 75% w/w, about 0.2% w/w to about 74% w/w, about 0.2% w/w to about 73% w/w, about 0.2% w/w to about 72% w/w, about 0.2% w/w to about 71% w/w, about 0.2% w/w to about 70% w/w, about 0.2% w/w to about 65% w/w, about 0.2% w/w to about 60% w/w, about 0.2% w/w to about 55% w/w, about 0.2% w/w to about 50% w/w, about 0.2% w/w to about 45% w/w, about 0.2% w/w to about 40% w/w, about 0.2% w/w to about 35% w/w, about 0.2% w/w to about 30% w/w, about 0.2% w/w to about 25% w/w, about 0.2% w/w to about 20% w/w, about 0.2% w/w to about 15% w/w, about 0.2% w/w to about 10% w/w, or about 0.2% w/w to about 5% w/w (% weight of the aggregation agent relative to the total weight of the ink particle).

In some embodiments of the ink particles of the invention, the ink particle can be made by any methods known in the art.

In some embodiments, the ink particle is a polymer microsphere encapsulating the coloring agent, which can be prepared using a wide variety of methods: solvent-in-emulsion evaporation, phase separation, coacervation, spray drying, crosslinking/gelation, hot melting, grinding, electro spraying, or polymerization (emulsion, suspension, dispersion, and precipitation). For polymerization techniques the starting material is unsaturated monomer molecules, which, upon chain-growth polymerization, will form the beads. For all the other techniques described afterward the starting material is already the polymer.

In some embodiments, the ink particle is prepared by emulsion techniques. There are two types of single emulsion techniques: oil-in-water (o/w) and water-in-oil emulsions (w/o). For example, the micro particulate carriers of natural polymers i.e. those of proteins and carbohydrates are prepared by these single emulsion techniques. The natural polymers are dissolved or dispersed in aqueous medium followed by dispersion in non-aqueous medium like oil. In the next step, the cross linking of the dispersed globule is carried out. The cross linking can be achieved either by means of UV light or heat or by using the chemical cross linkers. The chemical cross linking agents used are glutaraldehyde, formaldehyde, acid chloride etc. The nature of the surfactants used to stabilize the emulsion phases can greatly influence the size, size distribution, surface morphology, loading, dye/pigment release, and bio performance of the final multiparticulate product.

Double emulsion method of microspheres preparation involves the formation of the multiple emulsions or the double emulsion of type w/o/w and is best suited for water soluble coloring agents. This method can be used with both the natural as well as synthetic polymers. The aqueous dye/pigment solution is dispersed in a lipophilic organic continuous phase. The continuous phase is generally consisted of the polymer solution that eventually encapsulates of the dye/pigment contained in dispersed aqueous phase. The primary emulsion is subjected then to the homogenization or the sonication before addition to the aqueous solution of the poly vinyl alcohol (PVA). This results in the formation of a double emulsion. The emulsion is then subjected to solvent removal either by solvent evaporation or by solvent extraction.

In some embodiments, the ink particle is prepared by spray drying, freeze drying, or electrospray. In some embodiments, the ink particle is prepared by freeze drying. In some embodiments, the ink particle is prepared by freeze drying followed by cryomilling.

In some embodiments, the ink particle is prepared by spray drying. In spray drying technique, the polymer is first dissolved in a suitable volatile organic solvent such as dichloromethane, acetone, etc. The coloring agent in the solid form is then dispersed in the polymer solution with high-speed homogenization. This dispersion is then atomized in a stream of hot air. The atomization leads to the formation of the small droplets or the fine mist from which the solvent evaporates instantaneously leading the formation of the microspheres in a size range 200 nm-100 μm. The size can be manipulated by modifying several parameters, such as concentration of the polymer, solution flow rate, spraying rate, and drying temperature. Micro particles are separated from the hot air by means of the cyclone separator while the trace of solvent is removed by vacuum drying. One of the major advantages of this process is feasibility of operation under aseptic conditions.

In some embodiments, the ink particle is prepared by a dry coating process comprising a mechanical method that excludes any liquid solvent or binder solution. In some embodiments, the dry coating process is environmentally safe and cost-effective.

In some embodiments, the ink particle is prepared by Mechanofusion, hybridization, magnetic assisted impaction coating, theta-composer, rotating fluidized bed coating, pressure swing granulation, or high shear mixing.

In some embodiments, the ink particle is prepared by solvent evaporation. This process is carried out in a liquid manufacturing vehicle phase. The microcapsule coating is dispersed in a volatile solvent which is immiscible with the liquid manufacturing vehicle phase. A core material (e.g., the coloring agent) to be microencapsulated is dissolved or dispersed in the coating polymer solution. With agitation the core material mixture is dispersed in the liquid manufacturing vehicle phase to obtain the appropriate size microcapsule. The mixture is then heated if necessary to evaporate the solvent for the polymer of the core material is disperse in the polymer solution, polymer shrinks around the core. If the core material is dissolved in the coating polymer solution, matrix-type microcapsules are formed. The core materials may be either water soluble or water insoluble materials. Solvent evaporation involves the formation of an emulsion between polymer solution and an immiscible continuous phase whether aqueous (o/w) or non-aqueous.

In some embodiments, the ink particle is prepared by phase separation coacervation technique. This process is based on the principle of decreasing the solubility of the polymer in organic phase to affect the formation of polymer rich phase called the coacervates. In this method, the coloring agent particles are dispersed in a solution of the polymer and an incompatible polymer is added to the system which makes first polymer to phase separate and engulf the coloring agent particles. Addition of non-solvent results in the solidification of polymer. Poly lactic acid (PLA) microspheres have been prepared by this method by using butadiene as incompatible polymer. The process variables are very important since the rate of achieving the coacervates determines the distribution of the polymer film, the particle size and agglomeration of the formed particles. The agglomeration must be avoided by stirring the suspension using a suitable speed stirrer since as the process of microspheres formation begins the formed polymerize globules start to stick and form the agglomerates. Therefore the process variables are critical as they control the kinetic of the formed particles since there is no defined state of equilibrium attainment.

In some embodiments, the ink particle is prepared by solvent extraction. Solvent evaporation method is used for manufacturing of microparticles containing the coloring agent, involves removal of the organic phase by extraction of the non aqueous solvent. This method involves water miscible organic solvents as isopropanol. Organic phase can be removed by extraction with water. This process decreases the hardening time for the microspheres. One variation of the process involves direct incorporation of the coloring agent to the polymer organic solution. Rate of solvent removal by extraction method depends on the temperature of water, ratio of emulsion volume to the water and solubility profile of polymer.

In some embodiments, the ink particle is prepared by quasi emulsion solvent diffusion. A novel quasi-emulsion solvent diffusion method to manufacture the controlled release microspheres of drug with acrylic polymers has been reported in the literature. Microparticles can be manufactured by a quasi emulsion solvent diffusion method using an external phase containing distilled water and polyvinyl alcohol. The internal phase consists of dye/pigment, ethanol and polymer. The concentration of polymer is in order to enhance plasticity. At first, the internal phase is manufactured at 60° C. and then added to the external phase at room temperature. After emulsification process, the mixture is continuously stirred for 2 hours. Then the mixture can be filtered to separate the microparticles. The product is then washed and dried by vacuum oven at 40° C. for a day.

In some embodiments, the ink particle is prepared by a polymerization technique. The polymerization techniques conventionally used for preparing the microspheres are mainly classified as: normal polymerization or interfacial polymerization. Both are carried out in a liquid phase. Normal polymerization is carried out by using different techniques as bulk, suspension, precipitation, emulsion and micellar polymerization methods. In bulk, a monomer or a combination of monomers along with the initiator or catalyst is usually heated to initiate polymerization. Polymer so obtained may be molded as microspheres. The loading of the coloring agent may be done during the polymerization process. Suspension polymerization also referred as bead or pearl polymerization. It is carried out by heating the monomer or composition of monomers as droplets dispersion in a continuous aqueous phase. Droplets may also contain an initiator and other additives. Emulsion polymerization deviates from suspension polymerization as due to the presence initiator in the aqueous phase, which afterwards diffuses to the surface of micelles. Bulk polymerization has merits of formation of pure polymers.

Interfacial polymerization involves the reaction of various monomers at the interface between the two immiscible liquids to form a film of polymer that essentially envelops the dispersed phase. Ph-triggered microparticles are microparticles that release their payload when exposed to acidic conditions and, in some embodiments, are provided as a vehicle for dye/pigment release. Any dye/pigment can be encapsulated in a lipid-protein-sugar or polymer matrix with a pH-triggering agent to form microparticles. In some embodiments, the diameter of the pH-triggered microparticles ranges from 50 nm to 10 micrometers. The matrix of the ink particle can be prepared using any known lipid (e.g., DPPC), protein (e.g., albumin), or sugar (e.g., lactose). The matrix of the ink particle can also be prepared using any synthetic polymers, such as polyesters. The process of formulation includes providing an agent and contacting with a pH-triggering agent and component selected from lipid, proteins and sugars, and spray drying the resultant mixture to create microparticles. Typically, the pH-triggering agent is a chemical compound, including a polymer having a pKa less than 7. The pH-triggered microparticles release the encapsulated dye or pigment when exposed to an acidic environment.

In some embodiments, the ink particle is prepared by microfabrication. Microfabrication can be used to synthesize monodisperse microparticles. By generating highly monodisperse emulsion of polymer and the coloring agent droplets, controllable using a combination of driving pressures of two immiscible fluids and geometry of microchannels, microspheres containing dye/pigment with <5% mean deviation diameters can be obtained at a high throughput.

In some embodiments, the ink particle is prepared by a method comprising crosslinking/gelation. Sol-gel or gelation methods are used for fine-particle production. The gelation method uses a polymeric solution containing dye/pigment, starting from a sol state (colloidal solution) that evolves into a gel state (particles), which is extruded and submerged in a coagulation solution, which acts as a crosslinking agent of the polymer.

In some embodiments, the ink particle is prepared by electrohydrodynamic processes or electro spraying. Electrohydrodynamic processes or Electro spraying is a one-step technique which can generate narrow size distributions of submicrometric particles, with limited agglomeration of particles and high yields. The electro spraying process is that in which a polymer solution is loaded into a syringe and infused at a constant rate using a syringe pump through a small but highly charged capillary (e.g., a 16-26 gauge needle). The applied voltage used is typically up to + or −30 kV and the collector can be placed at a 7 to 30 cm distance from the capillary. Once the droplets have detached from the Taylor cone, the solvent evaporates, generating dense and solid particles, propelled towards the collector. In the context of dye/pigment loading, the dye/pigment is mixed to the polymer solution before electro spraying. Further, the size of the final product can be controlled by manipulating the governing factors such as the system, solution, instrumental and ambient parameters. The system parameters include the molecular weight and the micro structural feature of the polymer. The type and concentration of the polymer and solvent used, determine the solution properties namely pH, conductivity, viscosity and surface tension. The instrumental parameters include electrical potential applied, flow rate of the solution, distance between the tip of the needle and the collector and occasionally the nature of collector material. Additionally, the ambient conditions such as the temperature, humidity and air velocity in the process chamber together determine the rate of evaporation of the solvent from the electro sprayed product.

In some embodiments, the ink particle is prepared by a method comprising hot melting. This method has been also applied in pharmaceutical field to prepare sustained-release tablets and transdermal drug delivery systems. It can also be applied in ink particle preparation. This technique employs polymers with low melting point. The polymers are heated into the molten phase and then dispersed in a suitable dispersion medium containing dye/pigment and slowly cooled and fabricated into microsphere format. Microspheres with a SD between 1% and 5% have been reported.

In some embodiments, the ink particle is prepared by a method comprising precision particle fabrication (PPF) technology. PPF is a technology developed to produce uniform particles of a variety of materials and adapted for fabrication of controlled-release microparticle systems comprising biodegradable polymers. The main apparatus of PPF is based on passing a fluid containing the sphere-forming material(s) (i.e. biodegradable polymers) and any dye/pigment to be encapsulated through a small (10-100 pm) orifice to form a smooth, cylindrical stream. To break the stream into uniform droplets, the nozzle is acoustically excited by a piezoelectric transducer driven by a wave generator at a defined frequency. By employing an annular flow of a non-solvent phase, called the carrier stream, surrounding the polymer-dye/pigment jet to provide additional “drag” force, microparticle size and shape can be further controlled; particles even smaller than the nozzle openings can be generated.

The ink particle can comprise one or more stereoisomers, diastereomers and optical stereoisomers of any one or plurality of components of the ink particle described herein, as well as mixtures thereof. Additionally, stereoisomers, diastereomers, and optical stereoisomers of the components of the disclosure, and mixtures thereof, are within the scope of the disclosure. By way of non-limiting example, the mixture may include a racemate of coloring agent, polymer, or hydrogel, where the mixture may comprise unequal proportions of one particular stereoisomer of one or plurality of components in the ink particle over the others. Additionally, where applicable, a component of the ink particle can be provided as a substantially pure stereoisomers, diastereomers and optical stereoisomers (such as epimers).

Where applicable, the components of the ink particle described herein can be asymmetric (e.g., having one or more stereocenters). All stereoisomers, such as enantiomers and diastereomers, are intended to be included within the scope of the disclosure unless otherwise indicated. Components that contain asymmetrically substituted carbon atoms can be isolated in optically active or racemic forms. Methods of preparation of optically active forms from optically active starting materials are known in the art, such as by resolution of racemic mixtures or by stereoselective synthesis. Many geometric isomers of olefins, C—N double bonds, and the like can also be present in the compounds described herein, and all such stable isomers are contemplated in the present disclosure. Cis and trans geometric isomers of the component are also included within the scope of the disclosure and can be isolated as a mixture of isomers or as separated isomeric forms. Where the component is capable of stereoisomerism or geometric isomerism is designated in its structure or name without reference to specific R/S or cis/trans configurations, it is intended that all such isomers are contemplated.

Resolution of racemic mixtures of the component can be carried out by any of numerous methods known in the art, including, for example, fractional recrystallization using a chiral resolving acid which is an optically active, salt-forming organic acid. Suitable resolving agents for fractional recrystallization methods include, but are not limited to, optically active acids, such as the D and L forms of tartaric acid, diacetyitartaric acid, dibenzoyliartane acid, mandelic acid, malic acid, lactic acid, and the various optically active camphorsuifonic acids such as b-camphorsulfonic acid. Other resolving agents suitable for fractional crystallization methods include, but are not limited to. Stereoisomerically pure forms of -methyi-benzyl-amine (e.g., 5 and R forms, or diastereomerically pure forms), 2-phenylglycinoi, norephedrine, ephedrine, N-methyiephedrine, cyciohexyleihylamine, 1,2-diaminocyclohexane, and the like. Resolution of racemic mixtures can also be carried out by elution on a column packed with an optically active resolving agent (e.g., dinitrobenzoylphenylglycine). Suitable elution solvent compositions can be determined by one skilled in the art.

Any one or plurality of the ink particle components may also include tautomeric forms. Tautomeric forms result from the swapping of a single bond with an adjacent double bond together with the concomitant migration of a proton. Tautomeric forms include pro to tropic tautomers which are isomeric protonation states having the same empirical formula and total charge. Examples of prototropic tautomers include, but are not limited to, ketone-enol pairs, amide-imidic acid pairs, lactam-lactim pairs, amide-imidic acid pairs, enamine-imine pairs, and annular forms where a proton can occupy two or more positions of a heterocyclic system including, but not limited to, 1H- and 3H-imidazole, 1H-, 2H- and 4H-1,2,4-triazole, 1H- and 2H-isoindole, and 1H- and 2H-pyrazole, Tautomeric forms cars be in equilibrium or sterically locked into one form by appropriate substitution.

The ink particle of the invention may include salts, hydrates and solvate forms of any of the components in the particle. For instance, the polymer and the coloring agents may exist in anhydrous and/or non-solvated forms. Components can also include all isotopes of atoms occurring in the intermediates or final compounds. Isotopes include those atoms having the same atomic number but different mass numbers. For example, isotopes of hydrogen include tritium and deuterium.

The Ink Compositions of the Invention

The present invention provides an ink composition comprising plurality of ink particles of the invention.

In some embodiments of the ink composition of the invention, the composition is a powder. In some embodiments, the powder is substantially anhydrous. In some embodiments, water is present in the substantially anhydrous powder at a concentration of less than about 5% w/w (% weight of water relative to the total weight of the ink composition). In some embodiments, water is present in the substantially anhydrous powder at a concentration of less than: about 5% w/w, about 4% w/w, about 3% w/w, about 2% w/w, about 1% w/w, or about 0.5% w/w.

In some embodiments of the ink composition of the invention, the composition further comprises a liquid carrier. In some embodiments, the liquid carrier is a solution, suspension, gel, or emulsion. In some embodiments, the ink particles are partially or fully dissolved in the liquid carrier. In some embodiments, the ink particles are suspended in the liquid carrier.

In some embodiments of the ink composition of the invention, the composition is a solution, suspension, gel, or emulsion. In some embodiments, the composition is a homogenous or heterogeneous mixture.

In some embodiments, the liquid carrier comprises witch hazel. In some embodiments, witch hazel is present in the liquid carrier at a concentration of about 0.1% v/v to about 99% v/v (% volume of witch hazel relative to the total volume of the liquid carrier). In some embodiments, witch hazel is present in the liquid carrier at a concentration of about v/v, about 0.5% v/v, about 1% v/v, about 2% v/v, about 3% v/v, about 4% v/v, about 5% v/v, about 6% v/v, about 7% v/v, about 8% v/v, about 9% v/v, about 10% v/v, about 15% v/v, about 20% v/v, about 25% v/v, about 30% v/v, about 35% v/v, about 40% v/v, about 45% v/v, about 50% v/v, about 55% v/v, about 60% v/v, about 65% v/v, about 70% v/v, about 75% v/v, about 80% v/v, about 85% v/v, about 90% v/v, about 95% v/v, about 96% v/v, about 97% v/v, about 98% v/v, or about 99% v/v.

In some embodiments, the liquid carrier comprises glycerin. In some embodiments, glycerin is present in the liquid carrier at a concentration of about 0.1% v/v to about 10% v/v (% volume of glycerin relative to the total volume of the liquid carrier). In some embodiments, glycerin is present in the liquid carrier at a concentration of about 0.1% v/v, about 0.2% v/v, about 0.3% v/v, about 0.4% v/v, about 0.5% v/v, about 0.6% v/v, about 0.7% v/v, about 0.8% v/v, about 0.9% v/v, about 1% v/v, about 2% v/v, about 3% v/v, about 4% v/v, about 5% v/v, about 6% v/v, about 7% v/v, about 8% v/v, about 9% v/v, or about 10% v/v.

In some embodiments, the liquid carrier comprises sterile saline, buffer, water, ethanol, isopropanol, polyethylene glycol, polyol, or oil, or a mixture thereof.

In some embodiments, the liquid carrier comprises a buffer. In some embodiments, the buffer maintains the pH of the ink composition at a pH ranging from about 4 to about 7.5, from about 4 to a about 7, or from about 5 to about 7. In some embodiments, the buffer is a phosphate buffer or a triethanolamine buffer.

In some embodiments, the liquid carrier comprises sterile saline, phosphate buffered saline, water, ethanol, polyol (for example, glycerol, propylene glycol, and liquid polyethylene glycol, and the like), suitable mixtures thereof, or oil, such as vegetable oils.

In some embodiments, the liquid carrier comprises a humectant. As used herein, a “humectant” refers to any substance that promotes retention of moisture. In some embodiments, the humectant is polyhydric alcohols or glycerin. In some embodiments, the humectant is polyhydric alcohols such as ethylene glycol, propylene glycol, triethylene glycol, tetraethylene glycol, and sorbitol.

In some embodiments, the liquid carrier comprises a surfactant or a dispersant. In some embodiments, the liquid carrier comprises any surfactant or dispersant, in some embodiments those described herein. Where the liquid carrier comprises a surfactant or dispersant, the surfactant or dispersant of the liquid carrier can be the same as or different than the surfactant or dispersant of the outermost layer of an ink particle.

In some embodiments, the surfactant or dispersant of the outermost layer reduces or mitigates interparticle forces, e.g., ionic interactions, hydrophobic interactions, electrostatic interactions or Van der Waals forces. As such, in some embodiments, the surfactant or dispersant of the outermost layer reduces or mitigates ink particle aggregation where the ink particles are in the form of a power. In some embodiments, where the ink particles are in the form of a powder, a high frequency of rotation, e.g., about 2,000 rpm to about 10,000 rpm, is not necessary to dissolve or suspend the ink particles in a liquid carrier, and dissolution or suspension of the ink particles in the liquid carrier can be achieved using a significantly lower frequency of rotation, e.g., no greater than about 5,000 rpm, no greater than about 2,000 rpm, no greater than about 1,000 rpm, no greater than about 500 rpm, no greater than about 100 rpm or no greater than about 50 rpm.

In some embodiments, the liquid carrier comprises a stabilizer. As used herein, a “stabilizer” refers to a substance that prevents, inhibits or retards degradation. See, e.g., Concise Chemical and Technical Dictionary, Fourth Enlarged Edition, Bennet, Chemical Publishing Co., NY, N.Y. (1986).

In some embodiments of the ink composition of the invention, the ink particles are present in the liquid carrier at a concentration of from about 400 mg/mL to about 800 mg/mL. In some embodiments, the ink particles are present in the liquid carrier at a concentration of about 100 mg/mL, about 150 mg/mL, about 200 mg/mL, about 250 mg/mL, about 300 mg/mL, about 350 mg/mL, about 400 mg/mL, about 420 mg/mL, about 440 mg/mL, about 450 mg/mL, about 460 mg/mL, about 480 mg/mL, about 500 mg/mL, about 520 mg/mL, about 540 mg/mL, about 550 mg/mL, about 560 mg/mL, about 580 mg/mL, about 600 mg/mL, about 620 mg/mL, about 640 mg/mL, about 650 mg/mL, about 660 mg/mL, about 680 mg/mL, about 700 mg/mL, about 720 mg/mL, about 740 mg/mL, about 750 mg/mL, about 760 mg/mL, about 780 mg/mL, about 800 mg/mL, about 850 mg/mL, about 900 mg/mL, about 950 mg/mL, or about 1000 mg/mL. In some embodiments, the ink particles are present in the liquid carrier at a concentration of from about 350 mg/mL to about 800 mg/mL, from about 400 mg/mL to about 750 mg/mL, from about 350 mg/mL to about 600 mg/mL, or from about 400 mg/mL to about 600 mg/mL.

In some embodiments of the ink composition of the invention, the ink composition further comprises a pharmaceutically acceptable excipient, such as solvent, dispersant, dispersion media, diluent, suspension aid, surface active agent, isotonic agent, thickening or emulsifying agent, binder, lubricant, pH modifying agent, buffering agent, surfactant, isotonic agent, preservative, water soluble polymer (e.g., polyethylene glycols, polyvinyl pyrrolidone, dextran, and carboxymethyl cellulose), temperature responsive polymer (e.g. poly(N-isopropylacrylamide) and their copolymers, poly[2-(dimethylamino)ethyl methacrylate] (pDMAEMA), hydroxypropylcellulose, poly(vinylcaprolactame) and polyvinyl methyl ether) or combinations thereof. In some embodiments, the pharmaceutically acceptable excipient disclosed in Remington's The Science and Practice of Pharmacy, 21 st Edition, A. R. Gennaro, (Lippincott, Williams & Wilkins, Baltimore, Md., 2006) can be used. In some embodiments, water soluble polymer, temperature responsive polymer or a copolymer thereof, is present in the liquid carrier at a concentration of about 0.1% to about 50%, about 0.2% to about 50%, about 0.3% to about 50%, about 0.4% to about 50%, about 0.5% to about 50%, about 1% to about 50%, about 2% to about 50%, about 0.1% to about 50%, about 3% to about 50%, about 4% to about 50%, about 5% to about 50%, about 10% to about 50%, about 15% to about 50%, about 20% to about 50%, about 25% to about 50%, about 30% to about 50%, about 35% to about 50%, about 40% to about 50%, about 45% to about 50%, about 0.1% to about 49%, about 0.1% to about 48%, about 0.1% to about 47%, about 0.1% to about 46%, about 0.1% to about 45%, about 0.1% to about 40%, about 0.1% to about 35%, about 0.1% to about 30%, about 0.1% to about 25%, about 0.1% to about 20%, about 0.1% to about 15%, about 0.1% to about 10%, about 0.1% to about 5%, about 0.1% to about 4%, about 0.1% to about 3%, about 0.1% to about 2%, or about 0.1% to about 1% w/v of the liquid carrier (% weight of the water soluble polymer, temperature responsive polymer, or a copolymer thereof, relative to the volume of the liquid carrier).

In some embodiments, the preservative is prevents, inhibits or retards the growth of fungi or a microorganism. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal. In some embodiments, the liquid carrier comprises an antimicrobial agent.

In some embodiments of the ink composition of the invention, the ink composition further comprises a biocide. As used herein, a “biocide” is any chemical compound that inhibits, retards or prevents pathogen growth. In some embodiments, the biocide is an antibiotic. In some embodiments, the ink composition further comprises an antimicrobial agent: amikacin, anisomycin, apramycin, azithromycin, blasticidin S, brefeldin A, butirosin, chloramphenicol, chlortetracycline, clindamycin, clotrimazole, cycloheximide, demeclocycine, dibekacin, dihydrostreptomycin, doxycycline, duramycin, emetine, erythromycin, fusidic acid, G438, gentamicin, helvolic acid, hygromycin B, josamycin, kanamycin, kirromycin, lincomycin, meclocycline, mepartricin, midecamycin, minocycline, neomycin, netilmicin, nourseothricin, oleandomycin, oxytetraeycline, paromomycin, puromycin, rapamycin, ribostamycin, rifampicin, rifamyein, rosamicin, sisomicin, spectinomycin, spiramycin, streptomycin, tetracycline, thiaphenicol, thiostrepton, tobramycin, tunicamycin, tylosin, viomycin, virginiamycin, camptothecin, 10-deacetylbaccatin III, azacytidine, 7-aminoactinomycin D, 8-quinolinol, 9-dihydro-1, 3-acetylbaccatin III, aclarubicin, actinomycin D, actinomycin I, actinomycin V, bafilomycin A1, bleomycin, caprecmycin, chromomycin, cinoxacin, ciprofloxacin, cis-diammineplatinum(ii) dichloride, coumermycin A1, L(+)-lactic acid, cytochalasin B, cytochalasin D, dacarbazine, daunorubicin, distamycin A, doxorubicin, echinomycin, enrofloxacin, etoposide, flumequine, formycin, ganciclovir, metronidazole, mithramycin A, mitomycin C, nalidixic acid, nogalamycin, nonactin, novobiocin, ofloxacin, oxolinic acid, paclitaxel, phenazine, phleomycin, rebeccamycin, sinefungin, streptonigrin, streptozocin, succinylsulfathiazole, sulfadiazine, sulfadimethoxine, sulfaguanidine purum, sulfamethazine, sulfamonomethoxine, sulfanilamide, sulfaquinoxaline, sulfasalazine, sulfathiazole, trimethoprim, tubercidin, 5-azacytidine, formycin A, (+)-6-aminopenicillanic acid, 7-aminodesacetoxycephalosporanic acid, amoxicillin, ampicillin, azlocillin, bacitracin, carbenicillin, cefaclor, cefamandole, cefazolin, cefmetazole, cefoperazone, cefotaxime, cefsulodin, ceftriaxone, cephalexin, cephalosporin C, cephalothin, cephradine, cloxacillin, D-cycloserine, dicloxacillin, D-penicillamine, econazole, ethambutol, lysostaphin, moxalactam, nafcillin, nikkomycin Z, nitrofurantoin, oxacillin, penicillin G, phenethicillin, phenoxymethylpenicillin acid, phosphomycin, pipemidic acid, piperacillin, ristomycin, vancomycin, 2-mercaptopyridine, 4-bromocalcimycin A23187, alamethicin, amphotericin B, calcimycin A23187, chlorhexidine, colistin, hydrocortisone, filipin, gliotoxin, gramicidin A, gramicidin D, ionomycin, lasalocid A, lanomycin, monensin, N-(6-aminohexyl)-5-chloro-1-naphthalenesulfonamide, narasin, nigericin, nisin, nystatin, pimaricin, polymyxin B, DL-penicillamine, polymyxin E, praziquantel, salinomycin, surfactin, valinomycin, (+)-usnic acid, miconazole, 1-deoxymannojirimycin, 2-heptyl-4-hydroxyquinoline-oxide, cordycepin, 1,10-phenanthroline, 6-diazo-5-oxo-L-norleucine, antimycin, antipain, ascomycin, azaserine, bafilomycin, cerulenin, chloroquine, mevastatin, concanamycin A, concanamycin C, cyclosporin A, furazolidone, fusaric acid, geldanamycin, gramicidin C, herbimycin A, indomethacin, lomefloxacin, mycophenolic acid, myxothiazol, netropsin, niclosamide, nikkomycin, methyl-deoxynolirimycin, oligomycin, piericidin A, radicicol, staurosporine, stigmatellin, sulfaguanidine, triacsin C, paraceisin, rifaximin, loracarbef, ertapenem, doripenem, imipenem, cilastatin, meropenem, cefadroxil, cefalotin, cefalothin, cefoxitin, cefprozil, cefuroxime, cefalexin, cefdinir, cefditoren, cefpodoxime, ceftazidime, ceftibulen, ceftizoxime, cefepime, ceftaroline fosamil, ceftobiprole, teicopianin, telavanein, daptomycin, clarithromycin, dirithromycin, roxithromycin, gatifloxacin, levofloxacin, moxifloxacin, norfloxacin, trovailoxacin, grepafloxacin, sparfloxacin, temafloxacin, mafenide, sulfacetamide, silver suladiazine, sulfamethizole, sulfamethoxazole, sulfisoxazole, sulfonam idochrysoidine, clofazimine, dapsone, ethionamide, isoniazid, pyrazinamide, rifabutin, rifapentine, arsphenamine, fosfomycin, mupiroein, platensimycin, quinupristin, dalfopristin, tigecycline, ceftazidime, tinidazoie, artemisinin, artestmate, quinine, sulfadoxine-pyrimethamine, hydroxychloroquine, amodiaquine, pyrimethamine, sulphadoxine, proguanil, mefloquine, atovaquone, primaquine, and halofantrine. In any of the above embodiments, the antimicrobial agent is chosen from gentamicin, imipenem, piperacillin, ceftazidime, aztreonam, ceftriaxone, ampicillin, ciprofloxacin, linezolid, daptomycin, and rifampicirs. In some embodiments, the antimicrobial agent chosen from anisomyein, apramycin, blasticidin S, brefeldin A, butirosin, chlortetracycline, clotrimazole, cycloheximide, demeclocycline, dibekacin, dihydrostreptomycin, duramycin, emetine, fusidic acid, G438, helvolic acid, hygromycin B, kanamycin, kirromycin, lincomycin, meclocycline, mepartricin, midecamycin, netilmicin, nitrofurantoin, nourseothricin, oleandomycin, paromomycin, puromycin, rapamycin, ribostamycin, rifampicin, rifamycin, rosamicin, spectinomycin, spiramycin, streptomycin, thiamphenicol, camptothecin, lO-deacetylbaccatin III, azacytidine, 7-aminoactinomycin D, 8-quinolinol, 9-dihydro-1,3-acetylbaccatin III, aclarubicin, actinomycin D, actinomycin I, actinomycin V, bafilomycin A1, bleomycin, capreomycin, chromomycin, cinoxacin, ciprofloxacin, cis-diammineplatinum(ii) dichloride, coumermycin A1, L(+)-lactic acid, cytochalasin B, cytochalasin D, dacarbazine, daunorubicin, distamycin A, doxorubicin, echinomycin, enrofloxacin, etoposide, flumequine, formycin, fumagillin, ganciclovir, gliotoxin, metronidazole, mithramycin A, mitomycin C, nalidixic acid, netropsin, nitrofurantoin, nogalamycin, nonactin, novobiocin, oxolinic acid, paclitaxel, phenazine, phleomycin, pipemidic acid, rebeccamycin, sinefungin, streptonigrin, streptozocin, succinylsulfathiazole, sulfadiazine, sulfadimethoxine, sulfaguanidine purum, sulfamethazine, sulfamonomethoxine, sulfanilamide, sulfaquinoxaline, sulfasalazine, sulfathiazole, tubercidin, 5-azacytidine, cordycepin, formycin A, (+)-6-aminopenicillanic acid, 7-aminodesacetoxycephalosporanic acid, amoxicillin, ampicillin, azlocillin, bacitracin, carbenicillin, cefaclor, cefamandole, cefazolin, cefmetazole, cefotaxime, cefsulodin, cephalexin, cephalosporin C, cephalothin, cephradine, cloxacillin, D-cycloserine, dicloxacillin, D-penicillamine, econazole, ethambutol, lysostaphin, moxalactam, nafcillin, nikkomycin Z, nitrofurantoin, oxacillin, penicillic, penicillin G, phenethicillin, phenoxymethylpenicillinic acid, phosphomycin, pipemidic acid, piperacillin, ristomycin, vancomycin, 2-mercaptopyridine, 4-bromocalcimycin A23187, alamethicin, amphotericin B, calcimycin A23187, chlorhexidine, clotrimazole, econazole, hydrocortisone, filipin, gliotoxin, gramicidin A, gramicidin C, ionomycin, lasalocid A, lonomycin A, onensin, N-(6-aminohexyl)-5-chloro-1-naphthalenesulfonamide, narasin, nigericin, nisin, nystatin, phenazine, pimaricin, DL-penicillamine, praziquantel, salinomycin, 2-heptyl-4-hydroxyquinolone N-oxide, 1,6-diazo-5-oxo-L-norleucine, 8-quinolinol, antimycin, antipain, ascomycin, azaserine, bafilomycin, cerulenin, chloroquine, cinoxacin, mevastatin, concanamycin A, concanamycin C, coumermycin A1, cyclosporin A, furazolidone, radicicol, rapamycin, staurosporine, sulfaguanidine, triacsin C, trimethoprim, cilastatin, meropenem, cefadroxil, levofloxacin, moxifloxacin, trovafioxaein, grepafolxacin, sparfioxacin, temafloxacin, sulfamethizole, sulfamethoxazole, sulfonamidochrysoidine, clofazimine, dapsone, ethionamide, isoniazid, pyrazinamide, rifabutin, rifapentine, arsphenamine, fosfomycin, mupirocin, platensimycin, quinuprislin, dall pristin, tigecycline, imidazole, artemistin, artesunate, quinine, sulfadoxine-pyrimetbamine, hydroxychloroquinine, amodiaquine, sulphadoxine, proguanil, mefloquine, atovaquone, primaquine, or halofantrine. In some embodiments, the antimicrobial agent is imipenem, piperacillin, aztreonam, ampicillin, linezolid, daptomycin, or rifampicin, or combinations thereof.

The amount of the antimicrobial agent can determined based upon known dosage amounts. In some embodiments, the ink composition comprises a therapeutically effective amount of the antimicrobial agent.

Examples of a binder include, but are not limited to, cellulose; polyacrylic acid; starch (e.g. cornstarch and starch paste); gelatin; sugars (e.g. sucrose, glucose, dextrose, dextrin, molasses, lactose, lactitol, mannitol,); natural and synthetic gums (e.g. acacia, sodium alginate, extract of Irish moss, panwar gum, ghatti gum, karaya gum, tragacanth gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, polyvinyl pyrrolidone), magnesium aluminum silicate (Veegum), and larch arabogalactan); alginates; polyethylene oxide; polyethylene glycol; inorganic calcium salts; silicic acid; polymethacrylates; waxes; water; alcohol; etc.; and combinations thereof. In some embodiments, the binder is carboxymethylcellulose; cellulose; ethylcellulose; hydroxypropylmethylcellulose; methylcellulose; karaya gum; tragacanth gum, polyacrylic acid; or polyvinylpyrrolidone.

Examples of a diluent include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Examples of a dispersant include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Examples of an emulsifying agent include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Pluronic® F 127, Poloxamer 188, cetrimonium bromide, cetylpyridinium chloride, benzalkonium chloride, docusate sodium, etc. and/or combinations thereof.

In some embodiments, one or more ingredients in the liquid carrier or the pharmaceutically acceptable excipient in the ink composition are approved for use in humans and for veterinary use. In some embodiments, ingredients in the liquid carrier or the pharmaceutically acceptable excipient is approved by United States Food and Drug Administration. In some embodiments, one or more ingredients in the liquid carrier or the pharmaceutically acceptable excipient are pharmaceutical grade. In some embodiments, one or more ingredients in the liquid carrier or the pharmaceutically acceptable excipient meet the standards of the United States Pharmacopoeia (USP), the European Pharmacopoeia (EP), the British Pharmacopoeia, and/or the International Pharmacopoeia, which are incorporated herein in their entireties.

In some embodiments of the ink compositions of the invention, the ink particles are homogenous and/or heterogeneous species in non-aggregated form at room temperature or from about 65 to about 75 degrees Fahrenheit in the ink composition. In some embodiments, the ink compositions comprise the ink particles of homogenous and/or heterogeneous species in non-aggregated form at room temperature or from about 65 to about 75 degrees Fahrenheit, but, when exposed to body temperature or from about 98 to about 100 degrees Fahrenheit, the particles aggregate. In some embodiments, the ink compositions comprise the ink particles of homogenous and/or heterogeneous species in non-aggregated form at room temperature or from about 65 to about 75 degrees Fahrenheit, but, when exposed to body temperature or from about 98 to about 100 degrees Fahrenheit, the particles aggregate. In some embodiments, the clustering or aggregation properties of the ink particles is externally controllable. For instance, an electrical, magnetic, and/or a mechanical force can be used to bring the ink particles closer together and/or cause the ink particles to separate. Thus, in some embodiments, the application of an electrical, magnetic, and/or a mechanical force to the ink particles causes the particles to exhibit a change in color and/or increase the rate of dispersion upon administration. The clustering or aggregation of ink particles as discussed herein is not limited to generally spherical aggregations. In some embodiments, the ink particles cluster onto a surface. In some embodiments, the ink particles are be aligned relative to the surface due to an analyte or other external force. In some embodiments, poly(N-isopropylacrylamide) can form a gel matrix with ink particles at body temperature, which can aid in aggregation of the particles.

In some embodiments, the ink particles comprise reaction entities that are not necessarily binding partners to an analyte. For instance, there may a first reaction entity in the shell and a second reaction entity in the core that reacts with the first reaction entity; when the particles or contents of cavities are brought together in some fashion (e.g., by exposure to an analyte or other chemical that is recognized by binding partners on each of the particles, by the application of an electrical, magnetic, and/or a mechanical force to bring the particles closer together, or biodegradation, etc.), the first and second reaction entities may react. As a specific example, the reaction between the first and second reaction entities may be an endothermic or an exothermic reaction; thus, when the particles are brought together, a temperature change can occur, which can be determined using conventional means. As another example, a reaction between the first and second reactants may cause the release of a material. In some cases, the material may be one that can be sensed by a subject (e.g., human), e.g., capsaicin, an acid, an allergen, or the like. Thus, the subject may sense the change as a change in temperature, pain, itchiness, swelling, or the like. In some embodiments, the exposure of a first reaction entity with a second reaction entity chemically modifies a coloring agent such that the color of the design may be altered.

In some embodiments, the ink particles are suspended in the liquid carrier, or the ink particles are contained within a matrix, e.g., a porous matrix that is or becomes accessible by interstitial fluid after delivery, or a hydrogel matrix, etc. For instance, the matrix may be formed from a biodegradable and/or biocompatible material such as polylactic acid, polyglycolic acid, poly(lactic-co-glycolic acid), etc., or other similar materials.

In some cases, the matrix may prevent or at least inhibit an immunological response by the subject to the presence of the particles, while allowing equilibration of analytes, etc. with the ink particles to occur, e.g., if the matrix is porous. For instance, the pores of a porous matrix may be such that immune cells are unable to penetrate, while proteins, small molecules (e.g., glucose, ions, dissolved gases, etc.) can penetrate. The pores may be, for instance, less than about 5 micrometers, less than about 4 micrometers, less than about 3 micrometers, less than about 2 micrometers, less than about 1.5 micrometers, less than about 1.0 micrometers, less than about 0.75 micrometers, less than about 0.6 micrometers, less than about 0.5 micrometers, less than about 0.4 micrometers, less than about 0.3 micrometers, less than about 0.1 micrometers, less than about 0.07 micrometers, and in other embodiments, or less than about 0.05 micrometers. The matrix may comprise, for example, biocompatible and/or biodegradable polymers such as polylactic and/or polyglycolic acids, polyanhydride, polycaprolactone, polyethylene oxide, polybutylene terephthalate, starch, cellulose, chitosan, and/or combinations of these, and/or other materials such as agarose, collagen, fibrin, or the like.

In some embodiments of the ink composition of the invention, after the ink composition is intradermally administered to the subject, the ink particles remain in the dermis for a predetermined period of time and then disappears. In some embodiments, after the ink composition is intradermally administered to the subject, the ink particles remain in the dermis for about 2 months to about 10 years. In some embodiments, after the ink composition is intradermally administered to the subject, the ink particles remain in the dermis for about 2 months, about 3 months, about 4 months, about 5 months, about 6 months, about 7 months, about 8 months, about 9 months, about 10 months, about 11 months, about 12 months, about 1.5 years, about 2 years, about 2.5 years, about 3 years, about 3.5 years, about 4 years, about 4.5 years, about 5 years, about 5.5 years, about 6 years, about 6.5 years, about 7 years, about 7.5 years, about 8 years, about 8.5 years, about 9 years, about 9.5 years, or about 10 years.

In some embodiments of the ink composition of the invention, after the ink composition is intradermally administered to the subject, the ink particles biodegrade over a period from about 2 months to about 60 months. In some embodiments, after the ink composition is intradermally administered to the subject, the ink particles biodegrade over a period of about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 20 months, about 22 months, about 24 months, about 6 months, about 28 months, about 30 months, about 32 months, about 34 months, about 36 months, about 38 months, about 40 months, about 42 months, about 44 months, about 46 months, about 48 months, about 50 months, about 52 months, about 54 months, about 56 months, about 58 months, or about 60 months.

In some embodiments of the ink composition of the invention, the ink composition has a bioabsorption profile or a biodegradation profile that exhibits a lag phase of about 2 months to about 12 months. In some embodiments, the ink composition has a bioabsorption profile or a biodegradation profile that exhibits a lag phase of about 2 months, about 3 months, about 4 months, about 5 months, about 6 months, about 7 months, about 8 months, about 9 months, about 10 months, about 11 months, about 12 months, about 13 months, about 14 months, or about 15 months. In some embodiments, the ink composition has a bioabsorption profile or a biodegradation profile that exhibits a lag phase of about 2 months to about 12 months, about 3 months to about 12 months, about 4 months to about 12 months, about 5 months to about 12 months, about 6 months to about 12 months, about 7 months to about 12 months, about 8 months to about 12 months, about 9 months to about 12 months, or about 10 months to about 12 months.

In some embodiments, the ink composition has a bioabsorption profile or a biodegradation profile that exhibits a lag phase of about 8 weeks, about 10 weeks, about 12 weeks, about 14 weeks, about 16 weeks, about 18 weeks, about 20 weeks, about 22 weeks, about 24 weeks, about 26 weeks, about 28 weeks, about 30 weeks, about 32 weeks, about 34 weeks, about 36 weeks, about 38 weeks, about 40 weeks, about 42 weeks, about 44 weeks, about 46 weeks, about 48 weeks, about 50 weeks, about 52 weeks, about 54 weeks, about 56 weeks, about 58 weeks, about 60 weeks, about 62 weeks, about 64 weeks, about 66 weeks, about 68 weeks, or about 70 weeks.

In some embodiments of the ink composition of the invention, the polymer of each of the ink particles is present in an amount effective to prevent absorption of one or more coloring agents for a time period of from about 2 months to about 12 months, when the ink composition is intradermally administered to a subject.

In some embodiments of the ink composition of the invention, the polymer is present in an amount that is effective to induce aggregation of the ink particles upon their incorporation in a subject's dermis or effective to prevent or inhibit phagocytosis of the coloring agent upon incorporation in a subject's dermis.

In some embodiments of the ink composition of the invention, after the ink composition is intradermally administered to the subject, the ink particles contained within the skin may be alterable by the administration of an electrical, magnetic, and/or a mechanical force to the subject. For instance, by applying such forces, the ink particles may be caused to cluster, which may result in a change in color. In some embodiments, the region of the skin can be altered without electrical, magnetic, or mechanical force and only by adsorption and/or degradation of the ink particles by natural processes.

In some embodiments of the ink composition of the invention, after the ink composition is intradermally administered to the subject, the ink particles are physically or chemically modified to remain in the dermis indefinitely. In some embodiments of the ink composition of the invention, after the ink composition is intradermally administered to the subject, the ink particles are susceptible to a specific externally applied energy source, such as thermal, sonic (ultrasound), light (e.g., laser light, infrared light, or ultraviolet light), electric, magnetic, chemical, enzymatic, mechanical, or any other type of energy or combination of energies. Treatment of the tattooed skin with the appropriate energy source can sufficiently alters the coloring agent in the tattoo physically or chemically, allowing its elimination and, thus, erasing the tattoo on demand.

In some embodiments of the ink compositions of the invention, the ink compositions further comprises an additive. In some embodiments of the ink compositions of the invention, the liquid carrier further comprises an additive. In some embodiments, the additive is succinylated gelatin, arabinogalactan, glutaraldehyde, or petroleum wax, or mixtures thereof. In some embodiments, the additive is poloxanele, poly(acrylic acid co-hypophosphorite) sodium salt, polyacrylamide, alginate/alginic acid, calcium caseinate, calcium polypectate, cellulose acetate phthalate, cellulose acetate trimellitate, chitosan, edible and natural waxes, fatty acids, fatty alcohols, gellan gums, hydroxy cellulose, hydroxy ethyl cellulose, hydroxy methyl cellulose, hydroxy propyl cellulose, hydro propyl ethyl cellulose, hydroxy propyl methyl cellulose phthalate, lipids, mono-, di- and triglycerides, pectins, phospholipids, polyalkyl(Ci6-C22) acrylate, polyethylene, oxidized polyethylene, polyethyleneimine reacted with 1,2-dichloroethane, polyoxyethylene(600)dioleate, polyoxyethylene(600)monoricinoleate, polyoxyethylene(23)lauryl ether, polyethylene glycol, polyethylene glycol(400)dioleate, polyethylene glycol(400)mono-& di-oleate, polyglycerol esters of fatty acids, polyisobutylene, polyglycerol phthalate ester of coconut oil fatty acids, polymaleic acid and/or its sodium salts, polyoxyethylene glycol(400)mono-& di-oleates, polyoxyethylene (23) lauryl ether, polyoxyethylene(40)monostearate, polyoxyethylene-polyoxypropylene block polymers, polyoxyethylene(20)sorbitan monooleate, polyoxyethylene (20) sorbitan monostearate, polyoxyethylene(2)sorbitan tristearate, polyoxypropylene glycol, polyvinyl acetate, polysorbate 80, polyvinylpolypyrrolidone, polyvinylpyrrolidone, or poly(20vinylpyridine-co-styrene). In some embodiments, the additive is wax, polyolefin, paraffin (e.g., Bayberry, spermaceti, Japan, Ross, etc.), triglyceride, phospholipid, fatty acid or ester thereof (e.g., lauric acid, palmitic acid, sorbitan monopalmitate, sorbitan monostearate, etc.), poly(vinyl palmitate), poly(hexadecyl acrylamide), poly(butyl acrylate), poly(hexadecyl acrylate), poly(octadecyl acrylate), poly(dodecene), poly(isobutene), poly(trimethyl glutarate), polyanhydrides, poly orthoesters, polyesters, polystyrene, polyurethane, polypropylene, polymethacrylate, polytetrafluoroethylene, ceramic, or glass.

The Kits of the Invention

The present invention provides a kit comprising a) an ink composition of the invention, and b) a liquid carrier.

The present invention further provides a kit comprising: a) an ink composition comprising plurality of ink particles, wherein each ink particle comprises a core comprising a coloring agent and a polymer, wherein the polymer is polycaprolactone (PCL), poly D-lactic acid (PDLA), poly L-lactic acid (PLLA), poly(lactic-co-glycolic acid), (PLGA), polyethylene glycol (PEG), polyethylene glycol-diacrylate (PEGDA), poly(sebacic anhydride) (poly(SA)), polyorthoester, aliphatic polyanhydride, aromatic polyanhydride, or a copolymer thereof; and b) a liquid carrier comprising a surfactant or a dispersant.

The present invention further provides a kit comprising: a) an ink composition comprising plurality of ink particles, wherein each ink particle comprises:
(i) a core comprising a coloring agent; and
(ii) a shell;
wherein the core or the shell comprises a polymer, wherein the polymer is polycaprolactone (PCL), poly D-lactic acid (PDLA), poly L-lactic acid (PLLA), poly(lactic-co-glycolic acid), (PLGA), polyethylene glycol (PEG), polyethylene glycol-diacrylate (PEGDA), poly(sebacic anhydride) (poly(SA)), polyorthoester, aliphatic polyanhydride, aromatic polyanhydride, or a copolymer thereof; and
b) a liquid carrier comprising a surfactant or a dispersant.

The liquid carrier in the kit of the invention is as described herein.

In some embodiments of the kits of the invention, the ink composition and the liquid carrier are contained in the same container. In some embodiments, the container is a syringe. In some embodiments, the syringe has two or more separate compartments or chambers. In some embodiments, the syringe is a dual-chamber syringe. In some embodiments, the ink composition is contained in one chamber of the dual-chamber syringe and the liquid carrier is contained in the other chamber of the dual-chamber syringe.

In some embodiments of the kits of the invention, the ink composition and the liquid carrier are contained in separate containers.

In some embodiments of the kits of the invention, the kits further comprise a needle effective intradermal administration, e.g., for tattoo application.

The Methods of the Invention

The present invention provides a method for tattooing a subject, comprising intradermally administering to the subject a cosmetically effective amount of the ink composition of the invention.

In some embodiments, the method provides a tattoo that partially or fully changes color.

The present invention further provides a method for tattooing a subject, comprising the steps of: a) admixing the ink composition and the liquid carrier of the kit of the invention to provide a tattoo ink; and b) intradermally administering a cosmetically effective amount of the tattoo ink to the subject.

In some embodiments, the ink particles or the ink compositions are administered into the dermis and/or epidermis layer of the skin. In some embodiments, the ink particles or the ink compositions are administered via a needle.

In some embodiments of the methods of the invention, the admixing occurs with a frequency generated by hand-shaking or hand-mixing. In some embodiments, the admixing occurs with a frequency of rotation ranging from about 100 rpm to about 10,000 rpm. In some embodiments, the frequency of rotation is no greater than about 3,500 rpm. In some embodiments, the frequency of rotation is no greater than about 2,000 rpm. In some embodiments, the frequency of rotation is no greater than about 1,000 rpm. In some embodiments, the frequency of rotation is no greater than about 1,500 rpm. In some embodiments, the frequency of rotation is no greater than about 1,000 rpm. In some embodiments, the frequency of rotation is no greater than about 800 rpm. In some embodiments, the frequency of rotation is no greater than about 600 rpm. In some embodiments, the frequency of rotation is no greater than about 500 rpm. In some embodiments, the frequency of rotation is no greater than about 400 rpm. In some embodiments, the frequency of rotation is no greater than about 200 rpm. In some embodiments, the frequency of rotation is no greater than about 100 rpm. In some embodiments, the frequency of rotation is no greater than about 50 rpm.

In some embodiments of the methods of the invention, the admixing occurs with a frequency of rotation that is generated by hand-shaking or hand-mixing. In some embodiments, the admixing occurs with a frequency of rotation that is generated by a benchtop vortex mixer. In some embodiments, the frequency of rotation ranges from about 100 to 3,200 rpm.

In some embodiments, admixing occurs within a syringe. In some embodiments, the syringe has two or more separate compartments or chambers. In some embodiments, the syringe is a dual-chamber syringe. In some embodiments, the ink composition is contained in one chamber of the dual-chamber syringe and the liquid carrier is contained in the other chamber of the dual-chamber syringe.

In some embodiments, admixing occurs at temperature higher than room temperature. In some embodiments, admixing occurs at about room temperature. In some embodiments, admixing occurs at a temperature in the range of from about 20° C. to about 40° C. In some embodiments, admixing occurs at a temperature in the range of from about 20° C. to about 25° C.

In some embodiments of the methods of the invention, the methods are performed by a tattoo artist. In some embodiments, the tattoo artist is licensed, if required by the respective jurisdiction.

In some embodiments of the methods of the invention, the methods are performed by the subjects themselves, e.g., self-tattooing or do-it-yourself tattoos.

In some embodiments of the methods of the invention, the methods are performed by a person who is not the subject or by a device.

In some embodiments of the methods of the invention, the methods can be performed using a tattoo gun, a tattoo needle, or a needle for tattoo application. In some embodiments, the tattoo needle is a needle for a stick-and-poke application.

Examples of devices useful to apply a tattoo include an electromagnetic coil tattooing machine (such as that disclosed in U.S. Pat. No. 4,159,659 to Nightingale); a rotary permanent cosmetics application machine (such as that disclosed in U.S. Pat. No. 5,472,449 to Chou); or any manual tattooing device (such as the sterile single-use device marketed by Softap Inc., San Leandro, Calif.).

The present invention further provides methods for making an ink composition, comprising admixing a plurality of ink particles and a liquid carrier. In some embodiments, the admixing occurs with a frequency of rotation that is no greater than about 5,000 rpm.

In some embodiments, the admixing occurs with a frequency of rotation that is no greater than about 3,500 rpm. In some embodiments, the admixing occurs with a frequency of rotation that is no greater than about 2,000 rpm. In some embodiments, the admixing occurs with a frequency of rotation that is no greater than about 1,500 rpm. In some embodiments, the admixing occurs with a frequency of rotation that is no greater than about 1,000 rpm. In some embodiments, the admixing occurs with a frequency of rotation that is no greater than about 800 rpm. In some embodiments, the admixing occurs with a frequency of rotation that is no greater than about 600 rpm. In some embodiments, the admixing occurs with a frequency of rotation that is no greater than about 500 rpm. In some embodiments, the admixing occurs with a frequency of rotation that is no greater than about 400 rpm. In some embodiments, the admixing occurs with a frequency of rotation that is no greater than about 200 rpm. In some embodiments, the admixing occurs with a frequency of rotation that is no greater than about 100 rpm. In some embodiments, the admixing occurs with a frequency of rotation that is no greater than about 50 rpm.

In some embodiments, the admixing occurs with a centripetal force of no greater than about 1,000 N. In some embodiments, the admixing occurs with a centripetal force of no greater than about 900 N. In some embodiments, the admixing occurs with a centripetal force of no greater than about 800 N. In some embodiments, the admixing occurs with a centripetal force of no greater than about 700 N. In some embodiments, the admixing occurs with a centripetal force of no greater than about 600 N. In some embodiments, the admixing occurs with a centripetal force of no greater than about 500 N. In some embodiments, the admixing occurs with a centripetal force of no greater than about 400 N. In some embodiments, the admixing occurs with a centripetal force of no greater than about 300 N. In some embodiments, the admixing occurs with a centripetal force of no greater than about 200 N. In some embodiments, the admixing occurs with a centripetal force of no greater than about 100 N. In some embodiments, the admixing occurs with a centripetal force of no greater than about 50 N. In some embodiments, the admixing occurs with a centripetal force of no greater than about 25 N. In some embodiments, the admixing occurs with a centripetal force of no greater than about 10 N. In some embodiments, the admixing occurs with a centripetal force of no greater than about 5 N.

In some embodiments, the admixing is achieved using a planetary centrifugal mixer. In some embodiments, the admixing is achieved using a vortex mixer. In some embodiments, the admixing is achieved by hand mixing. In some embodiments, the admixing by hand mixing occurs with a centripetal force of no greater than about 5 N.

In some embodiments, admixing occurs at temperature higher than room temperature. In some embodiments, admixing occurs at about room temperature. In some embodiments, admixing occurs at a temperature in the range from about 20° C. to about 40° C. In some embodiments, admixing occurs at a temperature in the range from about 20° C. to about 25° C.

Each reference (including, but not limited to, journal articles, U.S. and non-U.S. patents, patent application publications, international patent application publications, gene bank accession numbers, and the like) cited in the present application is incorporated herein by reference in its entirety.

What is claimed is:

1. An ink particle comprising:
(a) a core comprising a coloring agent and a polymer, wherein the polymer is polycaprolactone (PCL), poly D-lactic acid (PDLA), poly L-lactic acid (PLLA), poly(lactic-co-glycolic acid), (PLGA), polyethylene glycol (PEG), polyethylene glycol-diacrylate (PEGDA), poly(sebacic anhydride) (poly(SA)), polyorthoester, aliphatic polyanhydride, aromatic polyanhydride, or a copolymer thereof; and
(b) an outermost layer comprising a surfactant or a dispersant,
wherein the core further comprises an outer surface and the outermost layer at least partially covers the outer surface of the core.

2. An ink particle comprising:
(a) a core comprising a coloring agent;
(b) a shell; and
(c) an outermost layer comprising a surfactant or a dispersant,
wherein:
the core or the shell comprises a polymer, wherein the polymer is polycaprolactone (PCL), poly D-lactic acid (PDLA), poly L-lactic acid (PLLA), poly(lactic-co-glycolic acid), (PLGA), polyethylene glycol (PEG), polyethylene glycol-diacrylate (PEGDA), poly(sebacic anhydride) (poly(SA)), polyorthoester, aliphatic polyanhydride, aromatic polyanhydride, or a copolymer thereof;
the core further comprises an outer surface;
the shell at least partially covers the outer surface of the core;
the shell further comprises an outer surface; and
the outermost layer at least partially covers the outer surface of the shell.

3. The ink particle of claim 2, wherein the shell and the core comprise the polymer.

4. The ink particle of claim 3, wherein the polymer of the shell and of the core are the same.

5. The ink particle of claim 3, wherein the polymer of the shell and of the core are different.

6. The ink particle of claim 1 or 2, wherein the polymer is a block copolymer.

7. The ink particle of claim 6, wherein the block copolymer is a diblock copolymer or a triblock copolymer.

8. The ink particle of claim 1 or 2, wherein the polymer is present in the ink particle at a concentration of about 10% w/w to about 90% w/w.

9. The ink particle of claim 1 or 2, wherein the coloring agent is adsorbed to, physically entrapped by, ionically bonded to or covalently bonded to the polymer.

10. The ink particle of claim 2 or 3, wherein the coloring agent is adsorbed to, physically entrapped by, ionically bonded to or covalently bonded to the polymer of the core.

11. The ink particle of claim 2 or 3, wherein the coloring agent is adsorbed to, physically entrapped by, ionically bonded to or covalently bonded to the polymer of the shell.

12. The ink particle of claim 2 or 3, wherein the polymer of the shell is an aliphatic polyanhydride or an aromatic polyanhydride, wherein the aliphatic polyanhydride is poly(sebacic anhydride) and wherein the aromatic polyanhydride is poly[bis(p-carboxyphenoxy)methane)], poly[1,3-bis(p-carboxyphenoxy)propane)], poly[1,6-bis(p-carboxyphenoxy)hexane], poly[1,4-bis(hydroxyethyl)terephthalate-alt-ethyloxyphosphate], or poly[1,4-bis(hydroxyethyl)terephthalate-alt-ethyloxyphosphate]-co-1,4-bis(hydroxyethyl)terephthalate-co-terephthalate (80/20).

13. The ink particle of claim 2 or 3, wherein the polymer of the shell is a polyorthoester (POE), wherein the POE is POE I, POE II, POE III, or POE IV.

14. The ink particle of claim 2 or 3, wherein the polymer of the core is an aliphatic polyanhydride or an aromatic polyanhydride, wherein the aliphatic polyanhydride is poly(sebacic anhydride) and wherein the aromatic polyanhydride is poly[bis(p-carboxyphenoxy)methane)], poly [1,3-bis(p-carboxyphenoxy)propane)], poly[1,6-bis(p-carboxyphenoxy)hexane], poly[1,4-bis(hydroxyethyl)terephthalate-alt-ethyloxy phosphate], or poly[1,4-bis(hydroxyethyl)terephthalate-alt-ethyloxyphosphate]-co-1,4-bis(hydroxyethyl)terephthalate-co-terephthalate (80/20).

15. The ink particle of claim 2 or 3, wherein the polymer of the core is a polyorthoester (POE), wherein the POE is POE I, POE II, POE III, or POE IV.

16. The ink particle of claim 1 or 2, wherein the coloring agent has a molecular weight of about 5 Daltons to about 10×10⁶ Daltons.

17. The ink particle of claim 1 or 2, wherein the coloring agent is a dye, is a pigment, is fluorescent, or is phosphorescent.

18. The ink particle of claim 1 or 2, wherein the coloring agent is melanin, [Phthalocyaninato(2-)] copper, FD&C Red 40 (Food Red 17), FD&C Yellow 5, Nigrosin, Reactive Black 5, Acid Blue 113, Brilliant black BN Granular (Food Black 1), D&C Yellow 10, FD&C Blue 1 (Food Blue 2), FD&C Blue 2, Acid Black 1, Acid Black 24, Acid Black 172, Acid Black 194, Acid Black 210, Spirulina Extract Powder, Gardenia Yellow 98%, Gardenia Yellow 40%, Gardenia Black, Gardenia Blue, Gardenia Red, Cochineal/Carmine, Annatto, Beta carotene, D&C Orange 4, D&C Red 33, D&C Red 22, Ext D&C Violet 2, D&C Yellow 8, FD&C Green 3, FD&C Red 4, FD&C Yellow 6, FD&C Red 3, Ponceau 4R, Acid Red 52, Carmoisine, Amamath, Brown HT, Black PN, Green S, Patent Blue V, Tartrazine, Sunset Yellow, Quinolline Yellow, Erythrosine, Brilliant Blue, Indigo Carmine, D&C Green 5, D&C Red 17, D&C Red 21, D&C Red 27, D&C Yellow 11, D&C Violet 2, D&C Green 6, D&C Red 30, D&C Red 31, D&C Red 28, D&C Red 7, D&C Red 6, D&C Red 34, D&C Yellow 10, Fake of Carmoisine, Fake of Ponceau 4R, Fanchon Yellow, Toluidine Red, Fake of Acid red 52, Fake of Allura Red, Fake of Tartrazine, Fake of Sunset Yellow, Fake of Brilliant Blue, Fake of Erythrosine, Fake of Quinoline, Fake of Indigo Carmine, Fake Patent Blue V, Fake Black PN, Fithol Rubin B, Iron Oxide Red, Iron Oxide Yellow, Iron Oxide Black, Iron Blue, Titanium Dioxide, D&C Red 36, Carbon Black, Ultramarine Blue, Ultramarine Violet, Ultramarine Red/Pink, Chromium Oxide Green, Mica, Chromium Hydroxide Green, Talc, Manganese Violet, Iron Oxide Burgundy, Iron Oxide Sienna, Iron Oxide Tan, Iron Oxide Amber, Iron Oxide Brown-G, Iron Oxide Brown S, Sodium Copper Chlorophyllin, Caramel, Riboflavin, Canthaxanthin, Paprika, natural tumeric, D&C Green 8, Ext D&C Yellow 7, NOIR Brilliant BN, Ferric Ammonium Ferrocyanide, D&C Yellow 10 Fake, FD&C Yellow 5 Fake, FD&C Yellow 6 Fake, D&C Red 21 Fake, D&C Red 33 Fake, FD&C Red 40 Fake, D&C Red 27 Fake, D&C Red 28 Fake, FD&C Blue 1 Fake, D&C Red 30 Fake, D&C Red 36 Fake, D&C Red 6 Fake, D&C Red 7 Fake, or D&C Black 2, or a combination thereof.

19. The ink particle of claim 1 or 2, wherein the coloring agent is present in the ink particle at a concentration of about 10% w/w to about 70% w/w.

20. The ink particle of claim 1 or 2, wherein the coloring agent is present in the ink particle at a concentration of about 25% w/w to about 50% w/w.

21. The ink particle of claim 1 or 2, wherein the ink particle has a diameter ranging from about 10 nm to about 10 μm.

22. The ink particle of claim 1 or 2, wherein the ink particle has a diameter ranging from about 10 nm to about 100 μm.

23. The ink particle of claim 1, wherein the outermost layer covers from about 0.1% to about 99% of the outer surface of the core.

24. The ink particle of claim 1, wherein the outermost layer covers 100% of the outer surface of the core.

25. The ink particle of claim 2, wherein the outermost layer covers from about 0.1% to about 99% of the outer surface of the shell.

26. The ink particle of claim 2, wherein the outermost layer covers 100% of the outer surface of the shell.

27. The ink particle of claim 1, wherein the ink particle has a diameter (D) and a hydrodynamic diameter (Dh), wherein the Dh is greater than D by from about 1 nm to about 1 μm.

28. The ink particle of claim 2, wherein the ink particle has a diameter (D) and a hydrodynamic diameter (Dh), wherein Dh is greater than D by from about 1 nm to about 1 μm.

29. The ink particle of claim 1 or 2, wherein the surfactant or the dispersant is present in the ink particle at a concentration of about 0.1% w/w to about 70% w/w.

30. The ink particle of claim 1 or 2, wherein the surfactant is a non-ionic surfactant.

31. The ink particle of claim 30, wherein the non-ionic surfactant is poly(alkylene-oxide) block copolymer, oligomeric alkyl-ethylene oxide, alkyl-phenol poly-ethylene, sorbitan ester, or PEG-PLGA.

32. The ink particle of claim 30, wherein the non-ionic surfactant is polyoxyethylene-polyoxypropylene or polyoxyethylene-polyoxypropylene-polyoxyethylene.

33. The ink particle of claim 30, wherein the non-ionic surfactant is a poloxamer.

34. The ink particle of claim 33, wherein the poloxamer is poloxamer 188 or poloxamer 407.

35. The ink particle of claim 30, wherein the non-ionic surfactant is a poly(ethylene oxide)-poly(propylene oxide)-poly(ethylene oxide) triblock copolymer.

36. The ink particle of claim 35, wherein the poly(ethylene oxide)-poly(propylene oxide)-poly(ethylene oxide) (EO-PO-EO) triblock copolymer is EO₂PO₃₀EO₂; EO₆PO₃₄EO₆; EO₁₃PO₃₀EO₁₃; EO₁₉PO₂₉EO₁₉; EO₁₉PO₄₃EO₁₉; EO₂₅PO₄₀EO₂₅; EO₁₀₃PO₃₉EO₁₀₃; EO₁₇PO₆₀EO₁₇; EO₂₇PO₆₁EO₂₇; EO₂₇PO₅₆EO₂₇; EO₁₃₂PO₅₀EO₁₃₂; EO₁₉PO₆₉EO₁₉; or EO₉₇PO₆₉EO₉₇.

37. An ink composition comprising a plurality of ink particles, each of which is the ink particle of claim 1 or 2.

38. The ink composition of claim 5, wherein the composition is a powder.

39. The ink composition of claim 37, further comprising a liquid carrier.

40. The ink composition of claim 39, wherein the liquid carrier is a solution.

41. The ink composition of claim 39, wherein the liquid carrier comprises witch hazel.

42. The ink composition of claim 41, wherein witch hazel is present in the liquid carrier at a concentration of about 0.1% to about 99% v/v.

43. The ink composition of claim 39, wherein the liquid carrier comprises glycerin.

44. The ink composition of claim 43, wherein glycerin is present in the liquid carrier at a concentration of about 0.1% to about 10% v/v.

45. The ink composition of claim 39, wherein the liquid carrier comprises sterile saline, buffer, water, ethanol, isopropanol, polyethylene glycol, polyol, or oil, or a mixture thereof.

46. The ink composition of claim 39, wherein the liquid carrier comprises a humectant.

47. The ink composition of claim 39, wherein the liquid carrier comprises a surfactant or a dispersant.

48. The ink composition of claim 47, wherein the surfactant is a non-ionic surfactant.

49. The ink composition of claim 48, wherein the non-ionic surfactant is poly(alkylene-oxide) block copolymer, oligomeric alkyl-ethylene oxide, alkyl-phenol poly-ethylene, sorbitan ester, or PEG-PLGA.

50. The ink composition of claim 48, wherein the non-ionic surfactant is polyoxyethylene-polyoxypropylene or polyoxyethylene-polyoxypropylene-polyoxyethylene.

51. The ink composition of claim 48, wherein the non-ionic surfactant is a poloxamer.

52. The ink composition of claim 48, wherein the poloxamer is poloxamer 188 or poloxamer 407.

53. The ink composition of claim 48, wherein the non-ionic surfactant is a poly(ethylene oxide)-poly(propylene oxide)-poly(ethylene oxide) triblock copolymer.

54. The ink composition of claim 53, wherein the poly(ethylene oxide)-poly(propylene oxide)-poly(ethylene oxide) (EO-PO-EO) triblock copolymer is EO₂PO₃₀EO₂; EO₆PO₃₄EO₆; EO₁₃PO₃₀EO₁₃; EO₁₉PO₂₉EO₁₉; EO₁₉PO₄₃EO₁₉; EO₂₅PO₄₀EO₂₅; EO₁₀₃PO₃₉EO₁₀₃; EO₁₇PO₆₀EO₁₇; EO₂₇PO₆₁EO₂₇; EO₂₇PO₅₆EO₂₇; EO₁₃₂PO₅₀EO₁₃₂; EO₁₉PO₆₉EO₁₉; or EO₉₇PO₆₉EO₉₇.

55. The ink composition of claim 39, wherein the liquid carrier comprises an antimicrobial agent.

56. The ink composition of claim 55, wherein the antimicrobial agent is benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, or thimerosal.

57. The ink composition of claim 39, wherein the ink particles are present in the liquid carrier at a concentration of from about 400 mg/mL to about 800 mg/mL.

58. The ink composition of claim 39, having a bioabsorption profile or a biodegradation profile that exhibits a lag phase of about 2 months to about 12 months.

59. The ink composition of claim 39, wherein the polymer is present in an amount that is effective to induce aggregation of the ink particles upon their incorporation in a subject's dermis or effective to prevent or inhibit phagocytosis of the coloring agent upon incorporation in a subject's dermis.

60. A kit comprising:
a) the ink composition of claim 37; and
b) a liquid carrier.

61. A kit comprising:
a) an ink composition comprising plurality of ink particles, wherein each ink particle comprises a core comprising a coloring agent and a polymer, wherein the polymer is polycaprolactone (PCL), poly D-lactic acid (PDLA), poly L-lactic acid (PLLA), poly(lactic-co-glycolic acid), (PLGA), polyethylene glycol (PEG), polyethylene glycol-diacrylate (PEGDA), poly(sebacic anhydride) (poly(SA)), polyorthoester, aliphatic polyanhydride, aromatic polyanhydride, or a copolymer thereof; and
b) a liquid carrier comprising a surfactant or a dispersant.

62. A kit comprising:
a) an ink composition comprising a plurality of ink particles, wherein each ink particle comprises:
(i) a core comprising a coloring agent; and
(ii) a shell,
wherein the core or the shell comprises a polymer, wherein the polymer is polycaprolactone (PCL), poly D-lactic acid (PDLA), poly L-lactic acid (PLLA), poly(lactic-co-glycolic acid), (PLGA), polyethylene glycol (PEG), polyethylene glycol-diacrylate (PEGDA), poly(sebacic anhydride) (poly(SA)), polyorthoester, aliphatic polyanhydride, aromatic polyanhydride, or a copolymer thereof; and
b) a liquid carrier comprising a surfactant or a dispersant.

63. The kit of any one of claims 60-62, wherein the ink composition is a powder.

64. The kit of any one of claims 60-62, wherein the ink composition and the liquid carrier are contained in separate containers.

65. The kit of any one of claims 60-62, wherein the liquid carrier comprises witch hazel.

66. The kit of claim 65, wherein witch hazel is present in the liquid carrier at a concentration of about 0.1% to about 99% v/v.

67. The kit of any one of claims 60-62, wherein the liquid carrier comprises glycerin.

68. The kit of claim 67, wherein glycerin is present in the liquid carrier at a concentration of about 0.1% to about 10% v/v.

69. The kit of any one of claims 60-62, wherein the liquid carrier comprises sterile saline, buffer, water, ethanol, isopropanol, polyethylene glycol, polyol, or oil, or a mixture thereof.

70. The kit of any one of claims 60-62, wherein the liquid carrier comprises a humectant.

71. The kit of any one of claims 60-62, wherein the liquid carrier comprises an antimicrobial agent.

72. The kit of claim 71, wherein the antimicrobial agent is benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, or thimerosal.

73. The kit of any one of claims 60-62, further comprising a needle effective for tattoo application.

74. A method for tattooing a subject, comprising intradermally administering to the subject a cosmetically effective amount of the ink composition of claim 37.

75. The method of claim 74, wherein the method provides a tattoo that partially or fully changes color.

76. The method of claim 75, wherein the tattoo partially or fully changes color as the ink particles degrade.

77. A method for tattooing a subject, comprising the steps of:
a) admixing the ink composition and the liquid carrier of the kit of claim 60 to provide a tattoo ink; and
b) intradermally administering a cosmetically effective amount of the tattoo ink to the subject.

78. A method of tattooing a subject, comprising the steps of:
a) admixing the ink composition and the liquid carrier of the kit of claim 61 to provide a tattoo ink; and
b) intradermally administering a cosmetically effective amount of the tattoo ink to the subject.

79. A method of tattooing a subject, comprising the steps of:
a) admixing the ink composition and the liquid carrier of the kit of claim 62 to provide a tattoo ink; and
b) intradermally administering a cosmetically effective amount of the tattoo ink to the subject.

80. The method of any one of claims 77-79, wherein the admixing occurs with a frequency ranging from that generated by hand-shaking or hand-mixing to about 10,000 rpm.

81. The method of any one of claims 77-79, wherein the admixing occurs within a syringe.

82. A method for making the ink composition of claim 39, comprising admixing the plurality of ink particles and the liquid carrier.

83. The method of claim 82, wherein the admixing occurs with a frequency of rotation of no greater than about 5,000 rpm.

84. The method of claim 83, wherein the frequency of rotation is no greater than about 2,000 rpm.

85. The method of claim 83, wherein the frequency of rotation is no greater than about 1,000 rpm.

86. The method of claim 83, wherein the frequency of rotation is no greater than about 500 rpm.

87. The method of claim 83, wherein the frequency of rotation is no greater than about 100 rpm.

88. The method of claim 83, wherein the frequency of rotation is no greater than about 50 rpm.

89. The method of any one of claims 82-84, wherein the admixing is achieved using a planetary centrifugal mixer.

90. The method of claim any one of claims 82-88, wherein the admixing is achieved by hand mixing or using a vortex mixer.

91. The ink composition of claim 39, wherein the ink particles are suspended in the liquid carrier.

92. The method of claim 82, wherein the admixing suspends the ink particles in the liquid carrier.
